SEQUENCE LISTING (29 sequences)

SEQ ID NO: 1 - protein, 4 aa, synthetic construct
RGDS

SEQ ID NO: 2 - protein, 5 aa, synthetic construct
GRGDS

SEQ ID NO: 3 - protein, 5 aa, synthetic construct
GRGDY

SEQ ID NO: 4 - protein, 11 aa, synthetic construct
GGSGGRGDSPG

SEQ ID NO: 5 - protein, 10 aa, synthetic construct
RGDSPASSKP

SEQ ID NO: 6 - protein, 14 aa, synthetic construct
CGGNGEPRGDYRAY

SEQ ID NO: 7 - protein, 6 aa, synthetic construct
  MOD_RES 3: note = hydroxyproline
GFPGER

SEQ ID NO: 8 - protein, 6 aa, synthetic construct
  MOD_RES 3: note = hydroxyproline
GLPGER

SEQ ID NO: 9 - protein, 6 aa, synthetic construct
GASGER

SEQ ID NO: 10 - protein, 6 aa, synthetic construct
  MOD_RES 3: note = hydroxyproline
GRPGER

SEQ ID NO: 11 - protein, 6 aa, synthetic construct
  MOD_RES 3: note = hydroxyproline
GMPGER

SEQ ID NO: 12 - protein, 6 aa, synthetic construct
GLSGER

SEQ ID NO: 13 - protein, 6 aa, synthetic construct
  MOD_RES 3: note = hydroxyproline
GAPGER

SEQ ID NO: 14 - protein, 6 aa, synthetic construct
  MOD_RES 3: note = hydroxyproline
GFPGEK

SEQ ID NO: 15 - protein, 6 aa, synthetic construct
  MOD_RES 3: note = hydroxyproline
GLPGEK

SEQ ID NO: 16 - protein, 6 aa, synthetic construct
GASGEK

SEQ ID NO: 17 - protein, 6 aa, synthetic construct
  MOD_RES 3: note = hydroxyproline
GRPGEK

SEQ ID NO: 18 - protein, 6 aa, synthetic construct
  MOD_RES 3: note = hydroxyproline
GMPGEK

SEQ ID NO: 19 - protein, 6 aa, synthetic construct
GLSGEK

SEQ ID NO: 20 - protein, 6 aa, synthetic construct
  MOD_RES 3: note = hydroxyproline
GAPGEK

SEQ ID NO: 21 - protein, 6 aa, synthetic construct
  MOD_RES 3: note = hydroxyproline
GLPGEN

SEQ ID NO: 22 - protein, 6 aa, synthetic construct
GLKGEN

SEQ ID NO: 23 - protein, 35 aa, synthetic construct
  DOMAIN 1..35: note = Module C (ADF-4)
GSSAAAAAAAASGPGGYGPENQGPSGPGGYGPGGP

SEQ ID NO: 24 - protein, 35 aa, synthetic construct
  DOMAIN 1..35: note = Module Ccys
GSSAAAAAAAASGPGGYGPENQGPCGPGGYGPGGP

SEQ ID NO: 25 - protein, 35 aa, synthetic construct
  DOMAIN 1..35: note = Module Ck
GSSAAAAAAAASGPGGYGPKNQGPSGPGGYGPGGP

SEQ ID NO: 26 - protein, 280 aa, synthetic construct
  REPEAT 1..280: note = C8
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 27 - protein, 560 aa, synthetic construct
  REPEAT 1..560: note = C16
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 28 - protein, 1120 aa, synthetic construct
  REPEAT 1..1120: note = C32
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

SEQ ID NO: 29 - protein, 1680 aa, synthetic construct
  REPEAT 1..1680: note = C48
GSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGPGSSAAAAAAAASGPGGYGPENQGPSGPGGYGPGGP

(19) United States
(12) Patent Application Publication
(10) Pub. No.: US 2026/0295115 A1
(43) Pub. Date: Oct. 1, 2026

(54) METHOD FOR PRODUCING A COLLAGEN AND SILK POLYPEPTIDE COMPOSITE STRUCTURE

(71) Applicant: AMSILK GMBH, Neuried (DE)

(72) Inventors: Thomas SCHEIBEL, Bayreuth (DE); Kim Sarah KOECK, Bayreuth (DE); Vanessa Tanja TROSSMANN, Eckersdorf (DE)

(21) Appl. No.: 19/478,014

(22) PCT Filed: Apr. 25, 2024
(86) PCT No.: PCT/EP2024/061487

§ 371 Date: Oct. 23, 2025

(30) Foreign Application Priority Data
Apr. 25, 2023 (EP) 23169934.9

(51) Int. Cl. A61L 26/00; A61L 27/22; A61L 27/24; B33Y 70/00

(52) U.S. Cl. CPC A61L 26/0047; A61L 26/0033; A61L 27/225; A61L 27/24; B33Y 70/00

(57) ABSTRACT

The present invention relates to a method for producing a collagen and silk polypeptide composite structure. In addition, the present invention relates to a collagen and silk polypeptide composite structure and uses thereof.

### CROSS REFERENCE TO RELATED APPLICATIONS

This application is a U.S. National Stage of PCT/EP2024/061487, international filing date Apr. 25, 2024, which claims priority to European Application No. 23169934.9, filing date Apr. 25, 2023.

### REFERENCE TO SEQUENCE LISTING IN ELECTRONIC FORMAT

The contents of the electronic sequence listing (558-116-pct-sequence-listing.xml; Size: 35,971 bytes; and Date of Creation: Apr. 5, 2023) is herein incorporated by reference in its entirety.

The present invention relates to a method for producing a collagen and silk polypeptide composite structure. In addition, the present invention relates to a collagen and silk polypeptide composite structure and uses thereof.

### BACKGROUND OF THE INVENTION

One promising form of 3D printing is bioprinting. With bioprinting, organic or tissue-like structures are created that mimic natural tissue. Well-known areas of application are medicine, bioengineering, and food industry. However, the range of applications for bioprinting becomes wider and wider. The bioprinting technology uses a material called bioink.

Type I collagen is the predominant extracellular matrix (ECM) molecule in the body. It belongs to the group of fibril-forming collagens, which consists of three alpha-helices that form a triple-helical structure. Collagen as part of a bioink is known in the art. Current bioinks containing pure collagen have several shortcomings such as poor printability and relatively weak mechanical properties compared to polymer-based gels, stabilized by cross-linking. Most modern collagen-based bioinks have been developed from aqueous, acidic collagen solutions at concentrations below 5 mg/ml. They are not suitable for direct bioprinting without additional stabilization by chemical cross-linking. Direct 3D printing was only possible and successful for highly concentrated acidic collagen solutions, which show high viscosities and shear thinning behaviour. Despite the highly concentrated collagen solutions, the printed constructs exhibit low mechanical properties and require a basic environment that induces fibrillization and cross-linking of collagen molecules. This cross-linking reaction of collagen solution can be achieved at physiological conditions (neutral pH and 37° C.), where collagen molecules start to self-organize time-dependent into fibrils, and collagen forms a hydrogel. In contrast thereto, direct 3D printing of already self-assembled collagen hydrogels is not possible due to needle clogging and phase separation. High shear forces arise due to the high pressure of 1-2 bar, leading to the separation of the aqueous and the solid fibrillar phase. This drawback substantially hampers collagen application in 3D bioprinting.

In order to produce scaffolds with feasible mechanical properties and to decrease the degradation rate, printed collagen scaffolds were additionally treated using chemical cross-linking agents to obtain covalent bonds. Typical cross-linking strategies contain glutaraldehyde-(GA), 1-ethyl-3-(-3-dimethylaminopropyl) carbodiimide hydrochloride (EDC)/N-Hydroxysulfosuccinimide (NHS), genipin, Irgacure®, lithium-Phenyl-2,4,6-trimethylbenzoylphosphinate (LAP), ruthenium, formaldehyde (FA), epoxy-functional cross-linking agent, or combinations thereof.

However, cross-linking is usually cytotoxic and cross-linking agents must be thoroughly removed before the scaffolds can be used with living cells or as implants. In vitro and in vivo experiments specifically showed that chemical cross-linking methods altered the physiological wound healing process, even at low concentrations. High cross-linking densities were even associated with an enhanced macrophage response, reduced cell infiltration, increased expression of proinflammatory cytokines, chronic wounds, and delayed wound healing.

A widely used approach to improve the physicochemical properties of collagen constructs is the blending of the collagen solution with other natural or synthetic biomaterials to form double networks or the blending of the collagen solution with inorganic particles to form composite materials. For example, collagen was blended with chitosan to form collagen-chitosan constructs or collagen was blended with alginate to form collagen-alginate constructs. However, additional cross-linking using toxic substances was still necessary to obtain mechanically stable constructs, which have the already mentioned drawbacks.

Another approach to obtain collagen scaffolds with improved mechanical properties is by particle reinforcement, which relies on introducing synthetic polymers or mineral fillers. For example, hydroxyapatite (HA)-collagen scaffolds were prepared by freeze-drying a suspension of collagen fibrils and HA particles, compression molding, or precipitating HA within a freeze-dried collagen scaffold. However, these methods also required cross-linking of the collagen matrix and the mixtures were not 3D printable.

Another approach to obtain collagen scaffolds with a good resolution is the use of a viscoelastic or thermoreversible supporting bath composed of a microparticle slurry, or an isotonic solution bath that provides support during printing and is subsequently removed upon melting at 37° C. Some commercially available collagens bioinks can be printed in a stabilization bath containing gelatin, alginate, or agarose particles. However, the acidic pH of the collagen bioinks is not suitable for adding cells before printing, and cells can only be seeded onto the printed scaffold afterwards.

In view of the drawbacks described above, there is an urgent need for printed collagen structures which are mechanically stable, biodegradable, biocompatible, and non-toxic and for a printing method which produces these structures.

The present inventors generated for the first time a collagen and silk polypeptide (collagen-silk) composite structure which is mechanically stable. The collagen and silk polypeptide (collagen-silk) composite structure was produced without the need of chemical crosslinking. Specifically, the present inventors extruded a collagen solution directly into a silk polypeptide (precipitation) solution to overcome the limitations mentioned above. In this respect, it should be noted that the previously performed attempt of blending a collagen solution with a silk polypeptide solution to obtain a printable ink to generate mechanically stable constructs afterwards, was not successful, as the direct mixture of a collagen solution and a silk polypeptide solution resulted in the aggregation of collagen and silk polypeptides and in the formation of a physically crosslinked stable clot that made printing impossible. In contrast thereto, the direct printing of a collagen solution into a silk polypeptide (precipitation) solution resulted in the formation of collagen fibrils upon contact with the silk polypeptides. This precipitation allowed a layer-by-layer deposition of collagen in order to form a mechanically stable collagen and silk polypeptide (collagen-silk) composite structure with high shape fidelity. The use of an RGD-modified silk polypeptide variant in the silk polypeptide (precipitation) solution further resulted in a structure showing not only superior mechanical properties but also providing additional cell interaction sites. Moreover, the cell-friendly silk polypeptide solution could also contain cells, which were then homogenously integrated into the collagen and silk polypeptide (collagen-silk) composite structure during the printing process. Both, direct cell encapsulation as well as a subsequent cell seeding were possible. Subsequent addition of a phosphate-containing buffer further induced silk particle formation, which could strengthen fibril formation and resulted in a mechanically stable particle-reinforced collagen and silk polypeptide (collagen-silk) composite structure without harming the incorporated or loaded cells, if present.

In addition, the collagen and silk (collagen-silk) polypeptide composite structure produced by the present inventors is entirely biodegradable and biocompatible as well as non-toxic. It can, therefore, be used in medicine, tissue engineering such as skin tissue engineering and for biomedical applications.

### SUMMARY OF THE INVENTION

In a first aspect, the present invention relates a method for producing a collagen and silk (collagen-silk) polypeptide composite structure comprising the step of:
introducing a collagen solution into a silk polypeptide solution (precipitation solution),
thereby obtaining the collagen and silk (collagen-silk) polypeptide composite structure.

In a second aspect, the present invention relates to a collagen and silk (collagen-silk) polypeptide composite structure obtainable by the method according to the first aspect.

In a third aspect, the present invention relates to a collagen and silk (collagen-silk) polypeptide composite structure comprising collagen and
a silk polypeptide dispersed therein,
wherein the collagen and the silk polypeptide are directly linked/associated with each other (without (the need of) a separate cross-linker).

In a fourth aspect, the present invention relates to an implant comprising or consisting of the collagen and silk (collagen-silk) polypeptide composite structure according to the second aspect or third aspect.

In a fifth aspect, the present invention relates to a wound closure material comprising or consisting of the collagen and silk (collagen-silk) polypeptide composite structure according to the second aspect or third aspect.

In a sixth aspect, the present invention relates to the use of the collagen and silk (collagen-silk) polypeptide composite structure according to the second aspect or third aspect for tissue engineering, biomedical applications, or biofabrication.

In a seventh aspect, the present invention relates to the use of a combination of a collagen solution and a silk polypeptide solution for producing/forming a collagen and silk (collagen-silk) polypeptide composite structure.

In an eighth aspect, the present invention relates to a system for carrying out the method according to the first aspect, wherein the system comprises a processor capable of executing machine readable instructions when the machine-readable instructions are loaded into the system.

In a ninth aspect, the present invention relates to a computer program comprising machine-readable instructions which when loaded into the system according to the eight aspect cause the system to carry out the method according to the first aspect.

This summary of the invention does not necessarily describe all features of the present invention. Other embodiments will become apparent from a review of the ensuing detailed description.

### DETAILED DESCRIPTION OF THE INVENTION

### Definitions

Before the present invention is described in detail below, it is to be understood that this invention is not limited to the particular methodology, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

Preferably, the terms used herein are defined as described in “A multilingual glossary of biotechnological terms: (IUPAC Recommendations)”, Leuenberger, H. G. W, Nagel, B. and Kölbl, H. eds. (1995), Helvetica Chimica Acta, CH-4010 Basel, Switzerland).

Several documents are cited throughout the text of this specification. Each of the documents cited herein (including all patents, patent applications, scientific publications, manufacturer's specifications, instructions, GenBank Accession Number sequence submissions etc.), whether supra or infra, is hereby incorporated by reference in its entirety. Nothing herein is to be construed as an admission that the invention is not entitled to antedate such disclosure by virtue of prior invention.

In the following, the elements of the present invention will be described. These elements are listed with specific embodiments. However, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

The term “comprise” or variations such as “comprises” or “comprising” according to the present invention means the inclusion of a stated integer or group of integers but not the exclusion of any other integer or group of integers. The term “consisting essentially of” according to the present invention means the inclusion of a stated integer or group of integers, while excluding modifications or other integers which would materially affect or alter the stated integer. The term “consisting of” or variations such as “consists of” according to the present invention means the inclusion of a stated integer or group of integers and the exclusion of any other integer or group of integers.

The terms “a” and “an” and “the” and similar reference used in the context of describing the invention (especially in the context of the claims) are to be construed to cover both the singular and the plural, unless otherwise indicated herein or clearly contradicted by context.

The term “biomaterial”, as used herein, refers to a material that has been engineered to interact with biological systems for a medical purpose, either a therapeutic (treat, augment, repair, or replace a tissue function of the body) or a diagnostic one. The term “biomaterial”, as used herein, also refers to a material which has been adapted and used for printing, e.g. of three-dimensional objects. Some of the most notable bioengineered substances are usually stronger than the average bodily materials, including soft tissue and bone. These constituents can act as future substitutes, even improvements, for the original body materials. Collagen molecules and silk polypeptides are suitable biomaterials.

The term “bioprinting”, as used herein, refers to a technique which allows the generation of organic or tissue-like structures that mimic natural tissue. The term “bioprinting”, as used herein, also refers to an additive manufacturing process that combines biomaterials, optionally biomaterials and one or more additives, such as cells and/or growth factors.

The bioprinting technology uses a material called bioink. Similar to inkjet printing, the bioprinter prints/creates layer-by-layer assembled structures, specifically with a specific pore size and porosity that promote the restoration of defects of soft or hard tissues.

Another indisputable advantage of the bioprinting is that it allows creating personalized implants for the specific needs of a patient, taking the individual features of the patient into account at the same time. Moreover, the use of this technology allows building complex structures that are already colonized with cells at the moment of bioprinting.

As with tissue engineering, biological and biologically functional tissues are produced in the laboratory. A broad field of potential applications in medicine and bioengineering is opening up for this novel technology. The technology has already seen recent progress in the production of cartilage tissue for reconstruction and regeneration. The tissue engineering is preferably skin tissue engineering.

The term “three dimensional (3D) bioprinting”, as used herein, refers to the utilization of 3D printing like techniques to combine biomaterials, optionally biomaterials and one or more additives, such as cells and/or growth, to fabricate biomedical parts, e.g. to imitate natural tissue characteristics. Generally, 3D bioprinting can utilize a layer-by-layer method to deposit materials known as bioinks to create tissue-like structures that can later be used in various medical and tissue engineering fields.

Specifically, 3D bioprinting is an additive manufacturing process for the production of living tissue. For example, biomaterial solutions loaded with living cells are printed layer-by-layer to generate a precursor structure that can mature into functional, biological tissue in subsequent culturing steps. Compared to other biofabrication methods, such as cell colonization or molding, 3D bioprinting has several advantages that are important for the production of complex tissues. These include the spatially controlled and reproducible placement of different biomaterials and cells in a biological structure, as well as the production of patient-specific 3D geometries. Another advantage is the high degree of automation and digitization of the process.

The 3D bioprinting covers a broad range of bioprinting techniques. For example, extrusion-based printing is a very common technique within the field of 3D printing which entails extruding, or forcing, a continuous stream of liquid through a sort of orifice, often a nozzle or syringe. When it comes to extrusion based bioprinting, there are three main types of extrusion. These are pneumatic driven, piston driven, and screw driven types of extrusion. Each extrusion method has its own advantages and disadvantages. For example, pneumatic extrusion uses pressurized air to force a liquid bioink through a depositing agent. The air which is used to move the bioink must be free of contaminants. Air filters are commonly used to sterilize the air before it is used. Piston driven extrusion utilizes a piston connected to a guide screw. The linear motion of the piston squeezes material out of the nozzle. Screw driven extrusion uses an auger screw to extrude material. The rotational motion forces the material down and out of the nozzle. Screw driven devices also allow for the use of higher viscosity materials and provide more volumetric control.

The term “bioink”, as used herein, refers to a biomaterial which is printable or to a material which is biomaterial-based and which is printable. Bioinks are materials which can be used to produce engineered/artificial live tissue. These bioinks are often further composed of cells. The cells are sometimes used in tandem with additional material that envelopes/coats the cells. This additional material can be the biomaterial itself. The bioink must meet certain characteristics, including rheological, mechanical, biofunctional and biocompatibility properties, among others. Collagen molecules and silk polypeptides are suitable biomaterials which can be used as bioink.

The term “biofabrication”, as used herein, refers to the development and cultivation of bioartificial, cellular tissues using a variety of biotechnological, biomedical, and materials science and engineering methods, processes, and procedures. The present inventors used bioprinting, in particular 3D bioprinting, to fabricate a bioproduct, especially a collagen and silk (collagen-silk) polypeptide composite structure.

The term “tissue engineering”, as used herein, refers to a biomedical engineering discipline that uses a combination of cells, engineering, materials, methods, and suitable biochemical and physicochemical factors to restore, maintain, improve, or replace different types of biological tissues. Tissue engineering often involves the use of cells placed on tissue scaffolds in the formation of new viable tissue for a medical purpose but is not limited to applications involving cells and tissue scaffolds.

The term “self-assembly”, as used herein, refers to a process in which a disordered system of pre-existing polypeptides forms an organized structure or pattern as a consequence of specific, local interactions (e.g. van der Waals forces, hydrophobic interactions, hydrogen bonds, and/or salt-bridges, etc.) among the polypeptides themselves, without external direction or triggers, although external factors might influence speed and nature of self-assembly. This particularly means that when two or more disordered and/or unfolded polypeptides are brought into contact, they interact with each other and consequently form a three-dimensional structure. The change from a disordered system to an organized structure or pattern during self-assembly is characterized by a transition from a fluid state to a gel-like and/or solid state and a corresponding increase in viscosity. The transition from a fluid state to a gel-like state can be monitored, for example, by optical measurement or rheology. These techniques are known to the skilled person. The transition from a fluid state to a solid state can be monitored, for example, using optical methods. The polypeptides mentioned above are preferably silk polypeptides.

The term “polypeptide particles”, as used herein, refers to polypeptide structures which are formed as a consequence or result of polypeptide assembly. In the process of polypeptide assembly, multiple copies/units of polypeptides aggregate into a body or mass. The self-assembly process may be induced by adding potassium or calcium phosphate as external trigger. Due to the high phosphate ion concentration, the polypeptides interact with each and precipitate as particles. In the polypeptide particles, the different polypeptides are connected with or attached to each other via covalent (e.g. disulfide bridges) and/or non-covalent interactions (e.g. van der Waals forces, hydrophobic interactions, hydrogen bonds, and/or salt-bridges). The polypeptide particles mentioned above are preferably silk polypeptide particles.

The term “silk polypeptide”, as used herein, refers to a polypeptide which comprises repetitive units/repeating building blocks made of amino acids. Specifically, a silk polypeptide possesses large quantities of hydrophobic amino acids such as glycine or alanine. Especially, the highly repetitive amino acid sequences are located in the large core domain of the silk polypeptide.

Based on DNA analysis, it was shown that all silk polypeptides comprise chains of repetitive units which further comprise a limited set of distinct shorter peptide motifs. The expressions “peptide motif” and “consensus sequence” can be used interchangeably herein. Generally, the silk consensus sequences can be grouped into four major categories: GPGXX, GGX, Aₓ or (GA)ₙ and spacers. These categories of peptide motifs in silk proteins have been assigned structural roles. For example, it has been suggested that the GPGXX motif is involved in a β-turn spiral, probably providing elasticity. The GGX motif is known to be responsible for a glycine-rich 3₁-helix. Both GPGXX and GGX motifs are thought to be involved in the formation of an amorphous matrix that connects crystalline regions, thereby providing elasticity of the fiber. Alanine-rich motifs typically contain 6 to 9 residues and have been found to form crystalline β-sheets. The spacers typically contain charged groups and separate the iterated peptide motifs into clusters.

The silk polypeptide can perform self-assembly. Preferably, the silk polypeptide is a recombinant silk polypeptide. More preferably, the silk polypeptide is a spider silk polypeptide. Even more preferably, the spider silk polypeptide is a recombinant spider silk polypeptide. An exemplarily process for producing a silk polypeptide used herein is described in WO 2006/008163 and in WO 2011/120690.

The silk polypeptide, as used herein, can be fused to at least one cell adhesion mediating protein (CAMP) recognition sequence. Such a silk polypeptide can also be designated as silk polypeptide variant. This cell adhesion mediating protein (CAMP) recognition sequence (e.g. RGD) allows the binding of the silk polypeptide to CAMP proteins (e.g. integrins) which are present on the surface of cells and which are involved in the binding to other cells and/or to the extracellular matrix (ECM) in a process called cell adhesion. Due to the fusion of the silk polypeptide to the at least one CAMP recognition sequence, additional cell interaction sites are produced/provided which are advantageous, for example, in tissue engineering approaches.

CAMP recognition sequences occur naturally in CAMP-binding proteins, e.g. in fibronectin, fibrinogen, or vitronectin. CAMP recognition sequences which may be used in the present invention comprise or consists of an RGD-containing module or a non-RGD-containing module. The RGD sequence is the cell attachment site of a large number of extracellular matrix (ECM), blood, and cell surface proteins. Particularly, integrins recognize this sequence. The RGD sequence is, for example, naturally comprised in the bone sialoprotein, in collagen, decorsin, disintegrin, fibronectin, fibrinogen, fibrillin, prothrombin, thrombospondin, tenascin, vitronectin, or in the von Willebrand factor. Proteins with a CAMP recognition sequence which differs from RGD are, for example, collagen, cytotactin/tenascin-C, epiligrin, factor X, α-Chain of fibrinogen, γ-Chain of fibrinogen, invasin, laminin, matrix metalloproteinase-2, neutrophil inhibitory factor, osteopontin, plasminogen, spectrin, tenascin, or VCAM-1. The GER, GEN, and GEK sequences are, for example, cell attachment sites which are naturally comprised in collagens. Said sequences are particularly used by collagen-binding integrins.

Preferably, the at least one CAMP recognition sequence comprises a module containing RGD, GER, GEK, or GEN. More preferably, the CAMP recognition sequence comprises a module containing a linear or cyclic RGD. A CAMP recognition sequence which comprises a module containing a linear RGD is preferred in cases where the peptide is fused by genetic engineering to the self-assembling polypeptide. In contrast thereto, a CAMP recognition sequence which comprises a module containing a cyclic RGD is preferred in cases where the peptide is fused by chemical coupling to the self-assembling polypeptide.

The terms “polypeptide” and “protein” are used interchangeably in the context of the present invention.

The term “cell adhesion”, as used herein, refers to a mechanism which allows the coupling of cells to adjacent cells (cell-cell adhesion) and/or the coupling of cells to the surrounding ECM (cell-ECM adhesion). Cell adhesion is the basis for the establishment and development of multicellular organisms. Cell adhesion processes are important for cell morphogenesis and the development of tissues. Therefore, cell adhesion plays an important role during proliferation, migration, and/or differentiation. Particularly, the cell adhesion mechanism is a cellular process which includes wound healing, homeostasis, tumor growth, and metastasis.

Collagen is one main component of the extracellular matrix (ECM) in natural tissues and is, therefore, well suited as a biomaterial for tissue engineering. However, it has proven difficult to produce collagen scaffolds that can replicate the structure and function of tissues and organs. The present invention relates to a method to 3D-bioprint collagen into a precipitation bath comprising silk polypeptides such as C₁₆ yielding a composite with excellent mechanical properties. The silk polypeptide precipitation bath induced assembly of the collagen into fibrils, and subsequent addition of a phosphate containing buffer such as potassium phosphate buffer led to the formation of silk particles and stabilization of the collagen fibrils. The produced collagen-silk composite scaffolds showed an internal structure of homogeneously distributed and interacting collagen fibrils and spider silk particles with significantly better mechanical properties compared to plain collagen scaffolds. Further, enzymatic degradation assays of the scaffolds over a 7-day period showed higher stability of the collagen-silk scaffolds compared to plain collagen scaffolds in the presence of wound proteases. Using the silk polypeptide variant C₁₆-RGD further increased compressive stress and elastic modulus compared to that of the unmodified variant. In this respect, it is referred to the experimental section and to the Figures.

The term “collagen”, as used herein, refers to the main structural protein in the extracellular matrix. It is found in various connective tissues of the body such as cartilage, bones, tendons, ligaments, and skin. As the main component of connective tissue, it is the most abundant protein in mammals making up from 25% to 35% of the whole-body protein content. Preferably, the collagen is fibril-forming collagen specifically consisting of three alpha-helices that form a triple-helical structure. More preferably, the collagen is collagen of type I, II, III, V, and/or XI. Even more preferably, the collagen is collagen of type I and/or III, e.g. collagen of type I/III.

The term “collagen and silk (collagen-silk) polypeptide composite structure”, as used herein, refers to a structure comprising collagen and a silk (collagen-silk) polypeptide, wherein the silk polypeptide is dispersed in this structure and wherein the collagen and the silk polypeptide are directly linked/associated with each other (without (the need of) a separate cross-linker). Specifically, the collagen is comprised in form of collagen fibrils and/or the silk polypeptide is comprised in form of silk polypeptide particles in the structure.

The silk polypeptide particles have preferably a mean diameter of between 0.9 and 1.5 μm, more preferably of between 1.0 and 1.5 μm, e.g. 0.9, 1.0, 1.1, 1.2, 1.3, 1.4, or 1.5 μm, and/or the collagen fibrils have preferably a mean diameter of between 150 and 250 nm, more preferably of between 180 and 220 nm, e.g. 150, 160, 170, 180, 190, 200, 210, 220, 230, 240, or 250 nm.

More specifically, the collagen and the silk polypeptide are present at a ratio of between 40:60 and 60:40, e.g. 40:60, 41:59, 42:58, 43:57, 44:56, 45:55, 46:54, 47:53, 48:52, 49:51, 50:50, 51:49: 52:48, 53:47, 54:46, 55:45, 56:44, 57:43, 58:42, 59:41, and 60:40, in the structure. Even more specifically, the collagen and silk (collagen-silk) polypeptide composite structure is a 3D collagen and silk (collagen-silk) polypeptide composite structure, a collagen and silk (collagen-silk) polypeptide laminate, a collagen and silk (collagen-silk) polypeptide composite scaffold.

The collagen used in the above structures is preferably fibril-forming collagen specifically consisting of three alpha-helices that form a triple-helical structure. More preferably, the collagen is collagen of type I, II, III, V, and/or XI. Even more preferably, the collagen is collagen of type I and/or III, e.g. collagen of type I/III. The silk polypeptide used in the above structures is preferably a spider silk polypeptide.

In the method of the present invention, a collagen solution is extruded into a spider silk polypeptide solution to produce a collagen and silk (collagen-silk) polypeptide composite structure. The term “extrusion” in this context means the application of pressure to the collagen solution to force it through an opening, e.g. a needle, nozzle, or jet. The extrusion can be performed by hand using a simple syringe or by any appropriate automated system, for example, a pipe through which the collagen solution is driven in a replenishing flow, with a nozzle at the end via which the collagen solution extrudes into a silk polypeptide solution.

Preferably, the extrusion speed is (specifically a printer introducing the collagen solution moves with a speed of) between 0.1 mm/s and 20 mm/s, between 1 mm/s and 15 mm/s, between 2 mm/s and 10 mm/s, between 3 cm/s and 8 cm/s, between 4 mm/s and 6 mm/s, and more preferably at 5 mm/s. For example, the extrusion speed is (specifically a printer introducing the collagen solution moves with a speed of) at least 0.1, 0.5, 1, 2, 3, 4, 5, 6, 7, 8, 9, 10, 11, 12, 13, 14, 15, 16, 17, 18, 19, or 20 mm/s.

The extrusion of the collagen solution into the silk polypeptide solution may take place directly (without air gap) or indirectly (with air gap).

More preferably, the extrusion of the collagen solution into the silk polypeptide solution takes place directly

The collagen solution and/or silk polypeptide solution used to produce a collagen and silk (collagen-silk) polypeptide composite structure may comprise additive matter. In this way, a functionalized collagen and silk (collagen-silk) polypeptide composite structure can be produced. It is also possible to add the additive matter to the collagen and silk (collagen-silk) polypeptide composite structure after its formation.

The term “additive matter”, as used herein, refers to a any compound or material having a purpose that may be useful in the present invention, e.g. any compound or material that can be added to the (printed) collagen and silk (collagen-silk) polypeptide composite structure, or any compound or material that can be added to the collagen or silk polypeptide solutions disclosed herein and that can finally be printed. Preferably, the additive matter is selected from the group consisting of cells, beads, electricity conducting metals, crystallites, and biologically active agents, preferably pharmaceutical active compound.

The term “biologically active agent”, as used herein, refers to any physical, chemical, or biological substance which has a biologically effect and which may be used in the treatment, cure, prophylaxis, or prevention of a pathological condition, e.g. a disease or disorder, or which may be used to otherwise enhance physical, psychical or mental well-being.

Accordingly, the term “pharmaceutically active compound”, as used herein, includes any compound with therapeutic, curative, prophylactic, or preventative effect. The terms “pharmaceutically active compound” and “drug” can interchangeably be used herein. For example, the compound can be a compound that affects or participates in tissue growth, cell growth, cell differentiation, a compound that is able to invoke a biological action such as an immune response, or a compound that can play any other role in one or more biological processes.

### Embodiments of the Invention

The present invention will now be further described. In the following passages, different aspects of the invention are defined in more detail. Each aspect so defined may be combined with any other aspect or aspects unless clearly indicated to the contrary. In particular, any feature indicated as being preferred or advantageous may be combined with any other feature or features indicated as being preferred or advantageous, unless clearly indicated to the contrary.

Even at higher solution concentrations, (3D) printing of pure collagen solutions did not result in mechanically stable constructs. They could only be achieved upon chemical cross-linking using toxic, specifically cell-toxic, substances. The present inventors surprisingly found that the combination of collagen and silk polypeptides was advantageous for (3D) printing. The present inventors generated for the first time a collagen and silk polypeptide (collagen-silk) composite structure which is mechanically stable. The collagen and silk polypeptide (collagen-silk) composite structure was produced without the need of chemical crosslinking. Specifically, the present inventors extruded a collagen solution directly into a silk polypeptide (precipitation) solution to overcome the limitations mentioned above. In this respect, it should be noted that the previously performed attempt of blending a collagen solution with a silk polypeptide solution to obtain a printable ink to generate mechanically stable constructs afterwards was not successful, as the direct mixture of a collagen solution and a silk polypeptide solution resulted in the aggregation of collagen and silk polypeptides and in the formation of a physically crosslinked stable clot that made printing impossible. In contrast thereto, the direct printing of a collagen solution into a silk polypeptide (precipitation) solution resulted in the formation of collagen fibrils upon contact with the silk polypeptide solution. This precipitation allowed a layer-by-layer deposition of collagen in order to form a mechanically stable collagen and silk polypeptide (collagen-silk) composite structure with high shape fidelity. The use of an RGD-modified silk polypeptide variant in the silk polypeptide (precipitation) solution further resulted in a structure showing not only superior mechanical properties but also providing additional cell interaction sites. Moreover, the cell-friendly silk polypeptide solution could also contain cells, which were then homogenously integrated into the collagen and silk polypeptide (collagen-silk) composite structure during the printing process. Both, direct cell encapsulation as well as a subsequent cell seeding were possible. Subsequent addition of a phosphate-containing buffer further induced silk particle formation, which could strengthen fibril formation and resulted in a mechanically stable particle-reinforced collagen and silk polypeptide (collagen-silk) composite structure.

In addition, the collagen and silk (collagen-silk) polypeptide composite structure produced by the present inventors is entirely biodegradable and biocompatible as well as non-toxic. It can, therefore, be used in medicine, tissue engineering such as skin tissue engineering and for biomedical applications.

Thus, in a first aspect, the present invention relates to a method for producing a collagen and silk (collagen-silk) polypeptide composite structure comprising the step of:
introducing or depositing a collagen solution into a silk polypeptide solution (precipitation solution), thereby obtaining the collagen and silk (collagen-silk) polypeptide composite structure.

The collagen and silk (collagen-silk) polypeptide composite structure which is obtained with the above method is preferably biodegradable, biocompatible, and/or cell-friendly.

The method for producing a collagen and silk (collagen-silk) polypeptide composite structure can also be designated as (3D) (bio)printing method/process. The collagen solution which is introduced into the silk polypeptide solution (precipitation solution) can also be designated as (bio)ink.

The present inventors found that the introduction or deposition of a collagen solution into a silk polypeptide solution resulted in the precipitation of the collagen upon contact with the silk polypeptide solution. This precipitate is visible in form of collagen fibrils. In other words, collagen fibril formation occurs (immediately/directly) upon contact with the silk polypeptide solution. Thus, the silk polypeptide solution can also be designated as precipitation solution. The obtained collagen and silk polypeptide composite structure is (before post-treatment) composed of collagen fibrils and silk polypeptides dispersed therein.

The collagen precipitation, specifically the formation of collagen fibrils, is optically visible. Before contact with the silk polypeptide solution, the collagen solution is transparent (a transparent strand). Only after contact with the silk polypeptide solution does the collagen solution (the collagen strand) becomes whitish. The collagen solution itself is not fibrillar. Fibrillar structures are formed upon contact with the silk polypeptide solution.

In one preferred embodiment, the introduction or deposition of the collagen solution into the silk polypeptide solution encompasses the extrusion or printing of the collagen solution into the silk polypeptide solution.

When the collagen solution is extruded into the silk polypeptide solution, usually pressure is applied to the collagen solution to force it through an opening, e.g. a needle, nozzle, or jet. The extrusion can be performed by hand using a simple syringe or by any appropriate automated system, for example, a pipe through which the collagen solution is driven in a replenishing flow, with a nozzle at the end via which the collagen solution extrudes into the silk polypeptide solution.

Preferably, the extrusion speed is (particularly a printer introducing the collagen solution moves with a speed of) between 0.1 mm/s and 20 mm/s, between 1 mm/s and 15 mm/s, between 2 mm/s and 10 mm/s, between 3 cm/s and 8 cm/s, between 4 mm/s and 6 mm/s, and more preferably at 5 mm/s. For example, the extrusion speed is (particularly a printer introducing the collagen solution moves with a speed of) at least 0.1, 0.5, 1, 2, 3, 4, 5, 6, 7, 8, 9, 10, 11, 12, 13, 14, 15, 16, 17, 18, 19, or 20 mm/s. Specifically, the extrusion speed is (particularly a printer introducing the collagen solution moves with a speed of) between 0.1 and 20 mm/s and preferably at a speed of between 4 and 6 mm/s and between 0.8 and 1.2 bar.

The extrusion of the collagen solution into the silk polypeptide solution may take place directly (without air gap) or indirectly (with air gap).

It is also possible that the driving force for extrusion/printing is gravity.

Preferably a syringe with a 25 G needle is used. Alternatively, a syringe with a 14, 16, 20, 22, or 27 G needle may be used for the extrusion/printing process. In addition, nozzles with an inner diameter of 14, 16, or 20 G may be used for the extrusion process.

Needles made of steel or plastic and/or straight or tapered needles are possible.

The collagen solution is preferably introduced such as extruded into the silk polypeptide solution via a syringe, (an extrusion) nozzle (which is part of a printing/extrusion unit), or jet. For this introduction such as extrusion step, the silk polypeptide solution is preferably comprised in a container. The container may be a petri dish or culture plate.

The introduction such as extrusion of the collagen solution into the silk polypeptide solution specifically takes place directly, i.e. without an air gap. For this purpose, the syringe, the (extrusion) nozzle (which is part of a printing/extrusion unit), or jet carrying the collagen solution is sunk/extruded/moved into the silk polypeptide solution. Thereby, the syringe, (extrusion) nozzle (which is part of a printing/extrusion unit), or jet carrying the collagen solution is always below the liquid level of the silk polypeptide solution. Specifically, the syringe, (extrusion) nozzle (which is part of a printing/extrusion unit), or jet carrying the collagen solution is always below the liquid level of the silk polypeptide solution and close to the surface of the container or the previously extruded layer within the container.

The special feature of this method is that neither the collagen solution nor the silk polypeptide solution comprises a separate cross-linker molecule. In other words, the collagen and the silk polypeptide are directly linked/associated with each other (without (the need of) a separate cross-linker molecule). The collagen and silk (collagen-silk) polypeptide composite structure also does not need to be re-treated with linker molecules to induce subsequent crosslinking. In the past, printed collagen or collagen composite structures were additionally treated using chemical cross-linking agents to obtain covalent bonds. Typical cross-linking strategies contained glutaraldehyde-(GA), 1-ethyl-3-(-3-dimethylaminopropyl) carbodiimide hydrochloride (EDC)/N-Hydroxysulfosuccinimide (NHS), genipin, Irgacure®, lithium-Phenyl-2,4,6-trimethylbenzoylphosphinate (LAP), ruthenium, formaldehyde (FA), epoxy-functional cross-linking agent, or combinations thereof. This is not necessary anymore. The avoidance of cross-linking has the great advantage that no (cell) toxic substances are part of the composite structure. This is very important for any medical application.

In one another (alternatively or additionally) preferred embodiment,
the concentration of the collagen in the collagen solution is between 5 mg/ml and 20 mg/ml, more preferably between 10 mg/ml and 20 mg/ml, even more preferably between 12 mg/ml and 15 mg/ml, and still even more preferably 15 mg/ml, e.g. 5, 6, 7, 8, 9, 10, 11, 12, 13, 14, 15, 16, 17, 18, 19, or 20 mg/ml, and/or
the concentration of the silk polypeptide in the silk polypeptide solution is between 5 mg/ml and 40 mg/ml or between 5 mg/ml and 50 mg/ml, more preferably between 10 mg/ml and 30 mg/ml, even more preferably between 12 mg/ml and 25 mg/ml, and still even more preferably 20 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml.

Specifically, the collagen solution is an acidic collagen solution and the silk polypeptide solution has a physiological pH (a pH of about 7.5) or is alkaline (a pH between 6.5 and 8.5).

In one also (alternatively or additionally) preferred embodiment,
the pH of the collagen solution is between pH 2.0 and 4.0, more preferably between pH 2.5 and 3.5, and even more preferably pH 3.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and/or
the pH of the silk polypeptide solution is between pH 6.5 and 8.5, more preferably between pH 7.0 and 7.8, and even more preferably pH 7.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In one particularly preferred embodiment,
the concentration of the collagen in the collagen solution is between 5 mg/ml and 20 mg/ml, e.g. 5, 6, 7, 8, 9, 10, 11, 12, 13, 14, 15, 16, 17, 18, 19, or 20 mg/ml, and the pH of the collagen solution is between pH 2.0 and 4.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and/or
the concentration of the silk polypeptide in the silk polypeptide solution is between 5 mg/ml and 40 mg/ml or between 5 mg/ml and 50 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml, and the pH of the silk polypeptide solution is between pH 6.5 and 8.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In this respect, it should be noted that the increase in pH at the time the collagen solution is introduced into the silk polypeptide solution (precipitation solution) specifically leads to collagen fibril formation.

The collagen solution and/or the silk polypeptide solution may further comprise an additive matter, i.e. one or more additive molecules. In this way, the functionality of the collagen and silk (collagen-silk) polypeptide composite structure can be increased.

In one further (alternatively or additionally) preferred embodiment,
the additive matter is comprised in the collagen solution in an amount in the range of 0.01 wt %/vol to 10 wt %/vol, and more preferably in an amount in the range of 0.5 wt %/vol to 1 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol, and/or
the additive matter is comprised in the silk polypeptide solution in an amount in the range of 0.01 wt %/vol to 10 wt %/vol, and more preferably in an amount in the range of 0.5 wt %/vol to 1 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

In one further particularly preferred embodiment,
the concentration of the collagen in the collagen solution is between 5 mg/ml and 20 mg/ml, e.g. 5, 6, 7, 8, 9, 10, 11, 12, 13, 14, 15, 16, 17, 18, 19, or 20 mg/ml, the pH of the collagen solution is between pH 2.0 and 4.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and the collagen solution comprises an additive matter in an amount in the range of 0.01 wt %/vol to 10 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol, and/or
the concentration of the silk polypeptide in the silk polypeptide solution is between 5 mg/ml and 40 mg/ml or between 5 mg/ml and 50 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml, the pH of the silk polypeptide solution is between pH 6.5 and 8.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, and the silk polypeptide solution comprises an additive matter in an amount in the range of 0.01 wt %/vol to 10 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

By adding the additive matter into the collagen solution and/or silk polypeptide solution, the additive matter is encapsulated within the collagen and silk polypeptide composite structure. This addition ensures implementation, integration, and/or homogenous distribution of the additive matter within the collagen and silk polypeptide composite structure.

It does not matter whether the collagen solution or the silk polypeptide solution, or both comprise the additive matter. When the collagen solution comprises the additive matter, the collagen and the additive matter are simultaneously extruded into the silk polypeptide solution. When the silk polypeptide solution comprises the additive matter, the additive matter distributes between the collagen molecules/fibrils and the silk polypeptides when the collagen solution is introduced into the silk polypeptide solution.

In case of the collagen solution, the additive matter is preferably selected from the group consisting of beads, electricity conducting metals, crystallites, and biologically active agents, preferably pharmaceutical active compounds. In case of the silk polypeptide solution, the additive matter is preferably selected from the group consisting of cells, beads, electricity conducting metals, crystallites, and biologically active agents, preferably pharmaceutical active compounds.

The above-mentioned collagen solution is preferably an aqueous solution, e.g. water or an aqueous buffer, such as an aqueous acidic buffer, comprising collagen, and/or the above-mentioned silk polypeptide solution is preferably an aqueous solution, e.g. water or an aqueous buffer, such as an aqueous solution having a physiological pH (a pH of about 7.5), comprising silk polypeptides.

As mentioned above, the collagen solution is preferably introduced such as extruded into the silk polypeptide solution via a syringe, (an extrusion) nozzle (which is part of a printing/extrusion unit), or jet. For this introduction such as extrusion step, the silk polypeptide solution is preferably comprised in a container. The container is preferably a petri dish or al culture plate.

The extrusion of the collagen solution into the silk polypeptide solution may take place directly (without air gap) or indirectly (with air gap). The extrusion of the collagen solution into the silk polypeptide solution preferably takes place directly, i.e. without an air gap. For this purpose, the syringe, the (extrusion) nozzle (which is part of a printing/extrusion unit), or jet carrying the collagen solution is sunk/extruded/moved into the silk polypeptide solution. Thereby, the syringe, (extrusion) nozzle (which is part of a printing/extrusion unit), or jet carrying the collagen solution is always below the liquid level of the silk polypeptide solution.

In the method of the present invention, it is now preferred that the collagen solution is introduced layer-by-layer into the silk polypeptide solution. In other words, by introducing the collagen solution into the silk polypeptide solution, a collagen and silk polypeptide composite structure in a layer-by-layer fashion is preferably achieved.

In this respect, it is advantageous that
the syringe and/or the container are movable relative to each other in a predetermined pattern along the x-, y- and z-axis to form the collagen and silk (collagen-silk) polypeptide composite structure, particularly each layer of said structure, or
the (extrusion) nozzle (which is part of a printing/extrusion unit) and/or the container are movable relative to each other in a predetermined pattern along the x-, y- and z-axis to form the collagen and silk (collagen-silk) polypeptide composite structure, particularly each layer of said structure, or
the jet and/or the container are movable relative to each other in a predetermined pattern along the x-, y- and z-axis to form the collagen and silk (collagen-silk) polypeptide composite structure, particularly each layer of said structure.

For example, the formation of a collagen and silk (collagen-silk) polypeptide composite structure in a layer-by-layer fashion may take place as follows: Usually, a printer, in particular a printing head including a nozzle, may be moved either in z-direction and/or in x- or y-direction in order to obtain the respective collagen and silk (collagen-silk) polypeptide composite structure step by step. Alternatively or additionally, the container comprising the silk polypeptide solution may be moved in z-direction and/or in x- or y-direction. For the sake of completeness, it is indicated that the x-, y- and z-directions are in relation to a Cartesian coordinate system.

Specifically, the syringe, (extrusion) nozzle (which is part of a printing/extrusion unit), or jet is lowered to the bottom of the container. The syringe, (extrusion) nozzle (which is part of a printing/extrusion unit), or jet carrying the collagen solution is thereby below the liquid level of the silk polypeptide solution. The extrusion/printing then takes place from the bottom to the top.

First, the collagen solution is extruded/printed along the x-axis and y-axis into the silk polypeptide solution. The syringe, (extrusion) nozzle (which is part of a printing/extrusion unit), or jet is then lifted (along the z-axis) so that another layer can be printed along the x-axis and y-axis into the silk polypeptide solution. This process is repeated until the desired layer height and/or the desired number of layers is reached. During the whole extrusion/printing step, the liquid level is kept so that the syringe, (extrusion) nozzle (which is part of a printing/extrusion unit), or jet is always under liquid. Thereby, the collagen and silk (collagen-silk) polypeptide composite structure in a layer-by-layer fashion is formed. In cases where the printing is carried out indirectly, i.e. with air gap, the syringe, (extrusion) nozzle (which is part of a printing/extrusion unit), or jet is located above the surface of the silk polypeptide solution.

The printed layer can have any desired dimension that fits into the container in which it is printed or that fills the area of the print bed. Therefore, the construct can be several centimetres in size. For example, the dimensions of the printed layer can be adapted according to the desired specifications:

In one more preferred embodiment, the method comprises an incubation step for an incubation period. Usually, no further collagen solution is introduced into the silk polypeptide solution during the incubation period. In this respect, it should be noted that during the incubation period, an exchange/interaction between collagen and silk polypeptides takes place. While the collagen forms fibrillary structures, the silk polypeptides comprised in the silk polypeptide solution arranges themselves between these fibrillary collagen structures. This process leads to the generation of a mechanically stable collagen and silk (collagen-silk) polypeptide composite structure. In this respect, mechanically stable means that the extruded filament strands are connected with each other and are not dissolving any more when the sample is moved or exposed to vibrations. The mechanical properties increase with increasing incubation time and, thereby, the fibril formation is increased. However, the constructs are still semi-solid and soft.

Preferably,
the incubation period is for between 10 min and 5 hours, more preferably between 1 and 3 hours, and even more preferably for 2 hours, e.g. 10, 15, 20, 25, 30, 35, 40, 45, 50, 55 min, 1, 1.5, 2, 2.5, 3, 3.5, 4, 4.5, or 5 hours, and/or
the incubation step is carried out at a temperature of between 5° C. and 35° C., more preferably between 15° C. and 25° C., even more preferably at 20° C. (room temperature), e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C.

More preferably,
the incubation period is for between 10 min and 5 hours, e.g. 10, 15, 20, 25, 30, 35, 40, 45, 50, 55 min, 1, 1.5, 2, 2.5, 3, 3.5, 4, 4.5, or 5 hours, and
the incubation step is carried out at a temperature of between 5° C. and 35° C., e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C.

In one even more preferred embodiment, the method comprises a post-treatment step. It is preferred that during the post-treatment step, the collagen and silk (collagen-silk) polypeptide composite structure is exposed to a buffer solution (post-treatment buffer solution). Usually, the collagen and silk polypeptide composite structure is exposed to a buffer solution (post-treatment buffer solution) after expiration of the incubation period.

Preferably,
the collagen and silk (collagen-silk) polypeptide composite structure is exposed to the buffer solution for a time period of between 1 min and 24 hours, more preferably between 5 min and 12 hours (overnight), even more preferably between 10 min and 60 min, and still even more preferably for 30 min, e.g. 1, 2, 3, 4, 5, 6, 7, 8, 9, 10, 11, 12, 13, 14, 15, 20, 25, 30, 35, 40, 45, 50, 55 min, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours, and/or
the collagen and silk (collagen-silk) polypeptide composite structure is exposed to the buffer solution at a temperature of between 5° C. and 40° C., more preferably between 15° C. and 25° C., and even more preferably at 37° C., e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.

A temperature of 37° C. is particularly preferred. The exposure to the buffer solution at a temperature of 37° C. allows the use/addition of living cells.

More preferably,
the collagen and silk (collagen-silk) polypeptide composite structure is exposed to the buffer solution for a time period of between 1 min and 24 hours, e.g. 1, 2, 3, 4, 5, 6, 7, 8, 9, 10, 11, 12, 13, 14, 15, 20, 25, 30, 35, 40, 45, 50, 55 min, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours, and
the collagen and silk (collagen-silk) polypeptide composite structure is exposed to the buffer solution at a temperature of between 5° C. and 40° C., e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.

It is particularly preferred that the buffer solution comprises phosphate (i.e. is a phosphate-containing buffer). Said buffer solution may have a pH of between 7 and 8, e.g. pH 7, 7.1, 7.2, 7.3, 7.4, 7.5, 7.6, 7.7, 7.8, 7.9, or 8.0. Specifically, the buffer solution comprises between 0.5 M and 2.0 M phosphate, more specifically between 1.0 M and 1.5 M phosphate, and even more specifically 1.0 M phosphate, e.g. 0.5, 0.6, 0.7, 0.8, 0.9, 1.0, 1.1, 1.2, 1.3, 1.4, 1.5, 1.6, 1.7, 1.8, 1.9, or 2.0 M phosphate. Said buffer solution may have a pH of between 7 and 8, e.g. pH 7, 7.1, 7.2, 7.3, 7.4, 7.5, 7.6, 7.7, 7.8, 7.9, or 8.0. The phosphate may be potassium phosphate, sodium phosphate, or ammonium phosphate. A buffer solution comprising potassium phosphate is more particularly preferred.

In this post-treatment step, it is advantageous to replace the silk polypeptide solution by the buffer solution, specifically buffer solution comprising phosphate. The present inventors found that the post-treatment (in the phosphate-containing buffer) induces silk polypeptide particle formation and further strengthens the collagen fibril formation. Thus, after post-treatment, the collagen and silk (collagen-silk) polypeptide composite structure is composed of collagen fibrils and silk polypeptide particles dispersed therein. The present inventors specifically found that the post-treatment step (in the phosphate-containing buffer) is a step which triggers natural cross-linking between the collagen and silk polypeptide molecules. Especially, non-covalent and/or covalent bonds are formed. This form of natural cross-linking has, however, nothing to do with the chemical cross-linking induced by the addition of cross-linking agents such as glutaraldehyde-(GA), 1-ethyl-3-(-3-dimethylaminopropyl) carbodiimide hydrochloride (EDC)/N-Hydroxysulfosuccinimide (NHS), or genipin.

Thus, the post-treatment leads to the generation of particularly mechanically stable collagen and silk (collagen-silk) polypeptide composite structure. In this respect, particularly mechanically stable means that the constructs could be removed from the substrate (e.g. petri dish or cell culture pate) via a spatula or tweezer and could be transferred to new substrates or measuring devices without disintegration or destruction of the construct. All strands and layers are connected with each other increasing the stiffness of the composite scaffold.

Specifically, before the post-treatment with phosphate, the collagen molecules are present in the collagen and silk (collagen-silk) polypeptide composite structure as individual fibrils. The silk polypeptides itself are mainly present as soluble silk polypeptides, but also show β-sheet-rich assemblages, like fibrils. In this structure, only slight physical interactions between the collagen fibrils and the soluble silk polypeptides, partially pre-assembled, silk polypeptides, are detectable.

The post-treatment by incubating the collagen and silk (collagen-silk) polypeptide composite structure in phosphate buffer introduces a high concentration of phosphate ions into the system. On the one hand, the pH and the phosphate ions cause further collagen fibril assembly. With an increasing number of collagen fibrils, the stability of the construct increases. On the other hand, the phosphate leads to the precipitation of silk particles. These silk particles are physically connected with/bond to the collagen and themselves and exhibit tremendous mechanics. In addition, ion-ion interactions probably occur, which further leads to the improvement of the mechanics of the structure.

In this respect, it should be noted that the presence of cells as additive matter affects the incubation period and incubation temperature in the post-treatment step. The exposure to the buffer solution in case of cells as additive matter is preferably carried out over a time period of 30 min, otherwise between 10 and 60 min. Alternatively or additionally, the exposure temperature in case of cells as additive matter is preferably 37° C., otherwise between 15 to 25° C.

In one still even more preferred embodiment, the method further comprises the step of applying an additive matter onto the collagen and silk (collagen-silk) polypeptide composite structure. In this way, the functionality of the collagen and silk (collagen-silk) polypeptide composite structure can be further increased.

The silk polypeptide particles comprised in the composite structure produced with the method of the first aspect have preferably a mean diameter of between 0.9 and 1.5 μm, more preferably of between 1.0 and 1.5 μm, e.g. 0.9, 1.0, 1.1, 1.2, 1.3, 1.4, or 1.5 μm, and/or the collagen fibrils comprised in the composite structure produced with the method of the first aspect have preferably a mean diameter of between 150 and 250 nm, more preferably of between 180 and 220 nm, e.g. 150, 160, 170, 180, 190, 200, 210, 220, 230, 240, or 250 nm.

Preferably, the additive matter is applied onto the collagen and silk (collagen-silk) polypeptide composite structure in an amount in the range of 0.01 wt %/vol to 10 wt %/vol, and more preferably in an amount in the range of 0.5 wt %/vol to 1 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

More preferably, the additive matter is selected from the group consisting of cells, beads, electricity conducting metals, crystallites, and biologically active agents, preferably pharmaceutical active compounds.

In this way, the additive matter, i.e. the one or more additive molecules, is seeded onto the collagen and silk polypeptide composite structure.

The collagen and silk (collagen-silk) polypeptide composite structure obtained with the above method is preferably a 3D collagen and silk (collagen-silk) polypeptide composite structure, a collagen and silk (collagen-silk) polypeptide laminate, or a collagen and silk (collagen-silk) polypeptide composite scaffold. The collagen and silk (collagen-silk) polypeptide composite structure may be gel-shaped, e.g. a hydrogel, without the additional post-treatment step. After the post-treatment step, which leads to a particle-reinforcement, a stiffer scaffold remained.

In one specific embodiment, the method for producing a collagen and silk (collagen-silk) polypeptide composite structure comprises the steps of:
(i) introducing such as extruding a collagen solution into a silk polypeptide solution (precipitation solution) via a syringe, a (an extrusion) nozzle (which is part of a printing/extrusion unit), or jet, particularly in a layer-by-layer fashion, wherein the concentration of the collagen in the collagen solution is between 5 mg/ml and 20 mg/ml, e.g. 5, 6, 7, 8, 9, 10, 11, 12, 13, 14, 15, 16, 17, 18, 19, or 20 mg/ml, and the pH of the collagen solution is between pH 2.0 and 4.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and the concentration of the silk polypeptide in the silk polypeptide solution is between 5 mg/ml and 40 mg/ml or between 5 mg/ml and 50 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml, and the pH of the silk polypeptide solution is between pH 6.5 and 8.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, thereby obtaining the collagen and silk (collagen-silk) polypeptide composite structure,
(ii) incubating the collagen and silk (collagen-silk) polypeptide composite structure (obtained in step (i)) for an incubation period for between 10 min and 5 hours, e.g. 10, 15, 20, 25, 30, 35, 40, 45, 50, 55 min, 1, 1.5, 2, 2.5, 3, 3.5, 4, 4.5, or 5 hours, and at an incubation temperature of between 5° C. and 35° C., e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, or 35° C., and
(iii) post-treating the collagen and silk (collagen-silk) polypeptide composite structure (of step (ii)) in a phosphate-containing buffer solution (post-treatment buffer solution), particularly by replacing the silk polypeptide solution by the phosphate-containing buffer solution, for a time period of between 1 min and 24 hours, e.g. 1, 2, 3, 4, 5, 6, 7, 8, 9, 10, 11, 12, 13, 14, 15, 20, 25, 30, 35, 40, 45, 50, 55 min, 1, 1.5, 2, 2.5, 3, 3.5, 4, 4.5, 5, 6, 7, 8, 9, 10, 11, 12, 13, 14, 15, 16, 17, 18, 19, 20, 21, 22, 23, or 24 hours, and at a temperature of between 5° C. and 40° C., e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40° C.

Preferably, the collagen solution and/or the silk polypeptide solution in step (i) comprises an additive matter in an amount in the range of 0.01 wt %/vol to 10 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

More preferably, the phosphate-containing buffer solution comprises between 0.5 M and 2.0 M phosphate, e.g. 0.5, 0.6, 0.7, 0.8, 0.9, 1.0, 1.1, 1.2, 1.3, 1.4, 1.5, 1.6, 1.7, 1.8, 1.9, or 2.0 M phosphate.

Especially, the formation of a collagen and silk (collagen-silk) polypeptide composite structure in a layer-by-layer fashion takes place as follows: A printer, in particular a printing head including a nozzle, is moved either in z-direction and/or in x- or y-direction in order to obtain the respective collagen and silk (collagen-silk) polypeptide composite structure step by step. Alternatively or additionally, a container comprising the silk polypeptide solution is moved in z-direction and/or in x- or y-direction.

Specifically, the syringe, (extrusion) nozzle (which is part of a printing/extrusion unit), or jet comprising the collagen solution is lowered to the bottom of a container comprising the silk polypeptide solution. The syringe, (extrusion) nozzle (which is part of a printing/extrusion unit), or jet carrying the collagen solution is thereby below the liquid level of the silk polypeptide solution. The extrusion/printing then takes place from the bottom to the top. First, the collagen solution is extruded/printed along the x-axis and γ-axis into the silk polypeptide solution. The syringe, (extrusion) nozzle (which is part of a printing/extrusion unit), or jet is then lifted (along the z-axis) so that another layer can be printed along the x-axis and γ-axis into the silk polypeptide solution. This process is repeated until the desired layer height/the desired number of layers is reached. During the whole extrusion/printing step, the liquid level is kept so that the syringe, (extrusion) nozzle (which is part of a printing/extrusion unit), or jet is under liquid. Thereby, the collagen and silk (collagen-silk) polypeptide composite structure in a layer-by-layer fashion is formed.

The collagen, as used herein, is collagen which is capable of forming fibrils/fibrillary structures. Preferably, the collagen is fibril-forming collagen consisting of three alpha-helices that form a triple-helical structure. More preferably, the collagen is collagen of type I, II, III, V, and/or XI. Even more preferably, the collagen is collagen of type I and/or III, e.g. collagen of type I/III.

The silk polypeptide, as used herein, can be fused to at least one cell adhesion mediating protein (CAMP) recognition sequence. Such a silk polypeptide can also be designated as silk polypeptide variant. This cell adhesion mediating protein (CAMP) recognition sequence (e.g. RGD) allows the binding of the silk polypeptide to CAMP proteins (e.g. integrins) which are present on the surface of cells and which are involved in the binding to other cells and/or to the extracellular matrix (ECM) in a process called cell adhesion. Due to the fusion of the silk polypeptide to the at least one CAMP recognition sequence, additional cell interaction sites are produced/provided which are advantageous, for example, in tissue engineering such as skin tissue engineering approaches. Moreover, the combination of a silk polypeptide and at least one CAMP recognition sequence produces a more stable composite than when a silk polypeptide is used alone.

Specifically, the at least one CAMP recognition sequence comprises a module containing RGD, GER, GEK, or GEN.

More specifically, the at least one CAMP recognition sequence comprises a module containing a linear or cyclic RGD.

Even more specifically,
the module containing a linear RGD is selected from the group consisting of RGDS (SEQ ID NO: 1), GRGDS (SEQ ID NO: 2), GRGDY (SEQ ID NO: 3), GGSGGRGDSPG (SEQ ID NO: 4), RGDSPASSKP (SEQ ID NO: 5), and CGGNGEPRGDYRAY (SEQ ID NO: 6), or the module containing a cyclic RGD is selected from the group consisting of c(RGDfK), c(RGDfC), and c(RGDfE).

Alternatively or additionally, the CAMP recognition sequence comprising
i) a module containing GER is selected from the group consisting of GFOGER (SEQ ID NO: 7), GLOGER (SEQ ID NO: 8), GASGER (SEQ ID NO: 9), GROGER (SEQ ID NO: 10), GMOGER (SEQ ID NO: 11), GLSGER (SEQ ID NO: 12), and GAOGER (SEQ ID NO: 13),
ii) a module containing GEK is selected from the group consisting of GFOGEK (SEQ ID NO: 14), GLOGEK (SEQ ID NO: 15), GASGEK (SEQ ID NO: 16), GROGEK (SEQ ID NO: 17), GMOGEK (SEQ ID NO: 18), GLSGEK (SEQ ID NO: 19), and GAOGEK (SEQ ID NO: 20), and/or
iii) a module containing GEN is selected from the group consisting of GLOGEN (SEQ ID NO: 21) and GLKGEN (SEQ ID NO: 22).

The CAMP may be an integrin, a selectin, or a cadherin.

The silk polypeptide, as used herein, is preferably a recombinant silk polypeptide. The (recombinant) silk polypeptide may be a spider silk polypeptide, e.g. a major ampullate silk polypeptide such as a dragline silk polypeptide, a minor ampullate silk polypeptide, or a flagelliform silk polypeptide of an orb-web spider. Particularly, the silk polypeptide is a spider silk polypeptide. More particularly, the spider silk polypeptide is a recombinant spider silk polypeptide.

It is particularly preferred that the (recombinant) silk polypeptide is a polypeptide with an amino acid sequence which comprises or consists of at least 50%, 60%, 65%, 70%, 75%, 80%, 85%, or 90% multiple copies of repetitive units. It is more preferred that the silk polypeptide is a polypeptide with an amino acid sequence which comprises or consists of at least 95% multiple copies of repetitive units. Said repetitive units may be identical or different.

It is further particularly preferred that the (recombinant) silk polypeptide consists of between 40 to 4000 amino acids. It is more preferred that the (recombinant) silk polypeptide consists of between 100 to 3500 amino acids or between 200 to 2500 amino acids. It is even more preferred that the (recombinant) silk polypeptide consists of between 250 to 2000 amino acids.

It is also particularly preferred that the (recombinant) silk polypeptide comprises at least two identical repetitive units. For example, the (recombinant) silk polypeptide may comprise between 2 to 100 repetitive units, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99, or 100 repetitive units.

It is particularly more preferred that the repetitive units are independently selected from the group consisting of module C having an amino acid sequence according to SEQ ID NO: 23 or variants thereof, module C^{Cys} having an amino acid sequence according to SEQ ID NO: 24 or variants thereof, and module C^{Lys} having an amino acid sequence according to SEQ ID NO: 25 or variants thereof.

The module C variant differs from the reference module C from which it is derived by up to 1, 2, 3, 4, 5, 6, 7, 8, 9, or 10, 11, 12, 13, 14, or 15 amino acid changes in the amino acid sequence (i.e. substitutions, additions, insertions, deletions, N-terminal truncations and/or C-terminal truncations). Such a module variant can alternatively or additionally be characterized by a certain degree of sequence identity to the reference module from which it is derived. Thus, the module C variant has a sequence identity of at least 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, 96, 97, 98, 99% or even 99.9% to the respective reference module C. Preferably, the sequence identity is over a continuous stretch of at least 5, 10, 15, 18, 20, 24, 27, 28, 30, 34, or more amino acids, preferably over the whole length of the respective reference module C.

The sequence identity may be at least 80% over the whole length, may be at least 85% over the whole length, may be at least 90% over the whole length, may be at least 95% over the whole length, may be at least 98% over the whole length, or may be at least 99% over the whole length of the respective reference module C. Alternatively, the sequence identity may be at least 80% over a continuous stretch of at least 5, 10, 15, 18, 20, 24, 28, or 30 amino acids, may be at least 85% over a continuous stretch of at least 5, 10, 15, 18, 20, 24, 28, or 30 amino acids, may be at least 90% over a continuous stretch of at least 5, 10, 15, 18, 20, 24, 28, or 30 amino acids, may be at least 95% over a continuous stretch of at least 5, 10, 15, 18, 20, 24, 28, or 30 amino acids, may be at least 98% over a continuous stretch of at least 5, 10, 15, 18, 20, 24, 28, or 30 amino acids, or may be at least 99% over a continuous stretch of at least 5, 10, 15, 18, 20, 24, 28, or 30 amino acids of the respective reference module C.

A fragment (or deletion) variant of module C has preferably a deletion of up to 1, 2, 3, 4, 5, 6, 7, 8, 9, 10, 11, 12, 13, 14, or 15 amino acids at its N-terminus and/or at its C-terminus. The deletion can also be internally.

Additionally, the module C variant or fragment is only regarded as a module C variant or fragment within the context of the present invention, if the modifications with respect to the amino acid sequence on which the variant or fragment is based do not negatively affect the ability of the silk polypeptide to form a collagen and silk (collagen-silk) polypeptide composite structure. The skilled person can readily assess whether the silk polypeptide comprising a module C variant or fragment still has this property. In this respect, it is referred to the examples comprised in the experimental part of the present patent application.

Module C^{Cys} or C^{Lys} variants may also be encompassed by the present invention. Regarding the module C^{Cys} or C^{Lys} variants, the same explanations/definitions apply which have been made with respect to the module C variant (see above).

It is particularly even more preferred that the silk polypeptide comprises (C)ₘ, (C)ₘC^{Cys}, (C)ₘC^{Lys}, C^{Cys} (C)ₘ, C^{Lys} (C)ₘ, (C^{Cys})ₘ, or (C^{Lys})ₘ, wherein m is an integer of 2 to 96, e.g. 2, 3, 4, 5, 6, 7, 8, 9, 10, 11, 12, 13, 14, 15, 16, 17, 18, 19, 20, 21, 22, 23, 24, 25, 26, 27, 28, 29, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, 60, 61, 62, 63, 64, 65, 66, 67, 68, 69, 70, 71, 72, 73, 74, 75, 76, 77, 78, 79, 80, 81, 82, 83, 84, 85, 86, 87, 88, 89, 90, 91, 92, 93, 94, 95, or 96.

It is particularly most preferred that the silk polypeptide comprises C₂, C₄, C₆, C₈, C₁₆, C₃₂, C₄₈, (C)₂C^{Cys}, (C)₄C^{Cys}, (C)₆C^{Cys}, (C)₈C^{Cys}, (C)₁₆C^{Cys}, (C)₃₂C^{Cys}, (C)₄₈C^{Cys}, (C)₂C^{Lys}, (C)₄C^{Lys} (C)₆C^{Lys}, (C)₈C^{Lys}, (C)₁₆C^{Lys}, (C)₃₂C^{Lys}, (C)₄₈C^{Lys}, C^{Cys} (C)₂, C^{Cys} (C)₄, C^{Cys} (C)₆, C^{Cys} (C)₈, C^{Cys} (C)₁₆, C^{Cys} (C)₃₂, C^{Cys} (C)₄₈, C^{Lys} (C)₂, C^{Lys} (C)₄, C^{Lys} (C)₆, C^{Lys} (C)₈, C^{Lys} (C)₁₆, C^{Lys} (C)₃₂, C^{Lys} (C)₄₈, C^{Cys}₂, C^{Cys}₄, C^{Cys}₆, C^{Cys}₈, C^{Cys}₁₆, C^{Cys}₃₂, C^{Cys}₄₈, C^{Lys}₂, C^{Lys}₄, C^{Lys}₆, C^{Lys}₈, C^{Lys}₁₆, C^{Lys}₃₂, C^{Lys}₄₈, or C₁₆RGD.

The silk polypeptide C₈ has the amino acid sequence according to SEQ ID NO: 26 (8 times module C), the silk polypeptide C₁₆ (16 times module C) has the amino acid sequence according to SEQ ID NO: 27, the silk polypeptide C₃₂ (32 times module C) has the amino acid sequence according to SEQ ID NO: 28, and the silk polypeptide C₄₈ (48 times module C) has the amino acid sequence according to SEQ ID NO: 29.

Particularly, the above-described silk polypeptide consists exclusively of repetitive units. In other words, the silk polypeptide particularly does not comprise/is free of non-repetitive units. The only component that can additionally be present as part of the silk polypeptide is a tag or moiety, e.g. allowing easy transcription of said silk polypeptide in expression systems and/or allowing easy isolation of said silk polypeptide from the expression systems. Said tag may be an his tag or a flag tag.

The method of the first aspect can also be designated as (3D) bioprinting method. (3D) bioprinting is an additive manufacturing process for the production of living tissue. For example, a collagen and silk (collagen-silk) polypeptide composite structure loaded with living cells is printed layer-by-layer to generate a precursor structure that can mature into functional, biological tissue in subsequent culturing steps. Compared to other biofabrication methods, such as cell colonization or molding, (3D) bioprinting has several advantages that are important for the production of complex tissues. These include the spatially controlled and reproducible placement of different biomaterials and cells in a biological structure, as well as the production of patient-specific 3D geometries. Another advantage is the high degree of automation and digitization of the process.

Thus, with the method of the first aspect, a 3D-printed silk polypeptide particle reinforced collagen composite scaffold is produced. This scaffold can be used, for example, for tissue engineering such as skin tissue engineering.

In a second aspect, the present invention relates to a collagen and silk (collagen-silk) polypeptide composite structure obtainable by the method according to the first aspect. The collagen and silk (collagen-silk) polypeptide composite structure obtainable with the method according to the first aspect is preferably a 3D collagen and silk (collagen-silk) polypeptide composite structure, a collagen and silk (collagen-silk) polypeptide laminate, or a collagen and silk (collagen-silk) polypeptide composite scaffold. The collagen and silk (collagen-silk) polypeptide composite structure may be gel-shaped, e.g. a hydrogel, without the additional post-treatment step. After the post-treatment step, which leads to a particle-reinforcement, a stiffer scaffold remained.

Specifically, before the post-treatment with phosphate, the collagen molecules are present in the collagen and silk (collagen-silk) polypeptide composite structure as individual fibrils. The silk polypeptides itself are mainly present as soluble silk polypeptides, but also show β-sheet-rich assemblages, like fibrils. In this structure, only slight physical interactions between the collagen fibrils and the soluble silk polypeptides, partially pre-assembled, silk polypeptides, are detectable.

The post-treatment by incubating the collagen and silk (collagen-silk) polypeptide composite structure in phosphate buffer introduces a high concentration of phosphate ions into the system. On the one hand, the pH and the phosphate ions cause further collagen fibril assembly. With an increasing number of collagen fibrils, the stability of the construct increases. On the other hand, the phosphate leads to the precipitation of silk particles. These silk particles are physically connected with/bond to the collagen and themselves and exhibit tremendous mechanics. In addition, ion-ion interactions probably occur, which further leads to the improvement of the mechanics of the structure.

In addition, the collagen and silk (collagen-silk) polypeptide composite structure which is obtained with the method according to the first aspect is preferably biodegradable, biocompatible, and/or cell-friendly.

In a third aspect, the present invention relates to a collagen and silk (collagen-silk) polypeptide composite structure comprising
collagen and
a silk polypeptide dispersed therein,
wherein the collagen and the silk polypeptide are directly linked/associated with each other (without (the need of) a separate cross-linker).

The collagen and silk (collagen-silk) polypeptide composite structure are preferably biodegradable, biocompatible, and/or cell-friendly.

The special feature of this construct is that the collagen and the silk polypeptide are directly linked/associated with each other (natural cross-linking) and that no separate (chemical) cross-linker molecule is used (chemical cross-linking with molecules different from collagen/silk). The collagen and silk (collagen-silk) polypeptide composite structure also does not need to be re-treated with linker molecules to induce subsequent crosslinking. In the past, printed collagen or collagen composite structures were additionally treated using chemical cross-linking agents to obtain covalent bonds. Typical cross-linking strategies contained glutaraldehyde-(GA), 1-ethyl-3-(-3-dimethylaminopropyl) carbodiimide hydrochloride (EDC)/N-Hydroxysulfosuccinimide (NHS), genipin, Irgacure®, lithium-Phenyl-2,4,6-trimethylbenzoylphosphinate (LAP), ruthenium, formaldehyde (FA), epoxy-functional cross-linking agent, or combinations thereof. This is not necessary anymore. The avoidance of cross-linking has the great advantage that no (cell) toxic substances are part of the composite structure. This is very important for any medical application.

Preferably, the collagen and the silk polypeptide are present at a ratio of between 40:60 and 60:40, e.g. 40:60, 41:59, 42:58, 43:57, 44:56, 45:55, 46:54, 47:53, 48:52, 49:51, 50:50, 51:49: 52:48, 53:47, 54:46, 55:45, 56:44, 57:43, 58:42, 59:41, and 60:40, in the structure.

More preferably, the collagen is comprised in form of collagen fibrils in the structure (before post-treatment, see first aspect of the present invention).

Even more preferably, the collagen is comprised in form of collagen fibrils and the silk polypeptide is comprised in form of silk polypeptide particles in the structure (after post-treatment, see first aspect of the present invention).

Specifically, before the post-treatment with phosphate, the collagen molecules are present in the collagen and silk (collagen-silk) polypeptide composite structure as individual fibrils. The silk polypeptides itself are mainly present as soluble silk polypeptides, but also show β-sheet-rich assemblages, like fibrils. In this structure, only slight physical interactions between the collagen fibrils and the soluble silk polypeptides, partially pre-assembled, silk polypeptides, are detectable.

The post-treatment by incubating the collagen and silk (collagen-silk) polypeptide composite structure in phosphate buffer introduces a high concentration of phosphate ions into the system. On the one hand, the pH and the phosphate ions cause further collagen fibril assembly. With an increasing number of collagen fibrils, the stability of the construct increases. On the other hand, the phosphate leads to the precipitation of silk particles. These silk particles are physically connected with/bond to the collagen and themselves and exhibit tremendous mechanics. In addition, ion-ion interactions probably occur, which further leads to the improvement of the mechanics of the structure.

Still even more preferably, the composite structure comprises an additive matter, e.g. one or more additive molecules. The additive matter may be encapsulated and homogenously distributed within the collagen and silk (collagen-silk) polypeptide composite structure. Alternatively or additionally, the additive matter may also be coated/seeded onto the surface of the collagen and silk (collagen-silk) polypeptide composite structure. In other words, the additive matter may be comprised inside the composite structure and/or on the outside of the composite structure. Specifically, the additive matter is selected from the group consisting of cells, beads, electricity conducting metals, crystallites, and biologically active agents, such as pharmaceutical active compounds. More specifically, the additive matter are cells. Even more specifically, the composite structure comprises cells, wherein the cells are comprised inside the composite structure and/or on the outside of the composite structure.

The silk polypeptide particles comprised in the collagen and silk (collagen-silk) polypeptide composite structure have preferably a mean diameter of between 0.9 and 1.5 μm, more preferably of between 1.0 and 1.5 μm, e.g. 0.9, 1.0, 1.1, 1.2, 1.3, 1.4, or 1.5 μm, and/or the collagen fibrils comprised in the collagen and silk (collagen-silk) polypeptide composite structure have preferably a mean diameter of between 150 and 250 nm, more preferably of between 180 and 220 nm, e.g. 150, 160, 170, 180, 190, 200, 210, 220, 230, 240, or 250 nm.

The collagen and silk (collagen-silk) polypeptide composite structure is preferably a 3D collagen and silk (collagen-silk) polypeptide composite structure, a collagen and silk (collagen-silk) polypeptide laminate, or a collagen and silk (collagen-silk) polypeptide composite scaffold. The collagen and silk (collagen-silk) polypeptide composite structure may be gel-shaped, e.g. a hydrogel, without the additional post-treatment step. After the post-treatment step, which leads to a particle-reinforcement, a stiffer scaffold remained.

As to the preferred collagen molecules and/or silk polypeptides, it is referred to the first aspect of the present invention.

The collagen and silk (collagen-silk) polypeptide composite structure preferably has a layer-by-layer fashion.

The collagen and silk (collagen-silk) polypeptide composite structure is preferably biodegradable, biocompatible, and/or non-toxic.

The composite structure is particularly a 3D-printed silk polypeptide particle reinforced collagen composite scaffold. This scaffold can be used, for example, for tissue engineering such as skin tissue engineering.

In a fourth aspect, the present invention relates to an implant comprising or consisting of the collagen and silk (collagen-silk) polypeptide composite structure according to the first aspect or second aspect. The implant may be a skin implant, soft tissue (skin) implant, muscle implant, connective tissue (tendon and/or ligament) implant, cartilage implant, or bone implant.

The implant comprising or consisting of the collagen and silk (collagen-silk) polypeptide composite structure is preferably biodegradable, biocompatible, and/or cell-friendly.

In a fifth aspect, the present invention relates to a wound closure material comprising or consisting of the collagen and silk polypeptide composite structure according to the first aspect or second aspect.

The wound closure material comprising or consisting of the collagen and silk (collagen-silk) polypeptide composite structure is preferably biodegradable, biocompatible, and/or cell-friendly.

In a sixth aspect, the present invention relates to the use of the collagen and silk (collagen-silk) polypeptide composite structure according to the first aspect or second aspect for tissue engineering such as skin tissue engineering, biomedical applications, or biofabrication.

Tissue engineering is a biomedical engineering discipline that uses a combination of cells, engineering, materials, methods, and suitable biochemical and physicochemical factors to restore, maintain, improve, or replace different types of biological tissues.

In particular, tissue engineering (TE) has emerged as a useful approach to treat tissue damages caused by diseases and trauma, which has shown many advantages as compared to conventional treatment strategies. To afford desirable therapeutic outcome, scaffolds prepared from various kinds of biomaterials have been used for TE to accommodate sufficient amounts of cells and to control cell function. As a unique type of scaffolds, hydrogels have been frequently used for TE because of their similar 3D structures to the native extracellular matrix (ECM), as well as their tunable biochemical and biophysical properties to control cell functions such as cell adhesion, migration, proliferation, and differentiation.

Preferably, the biomedical applications are wound closure material, suture material, soft tissue (skin) material, muscle material, connective tissue (tendon and/or ligament) material, cartilage material, bone material, or bridging aid in peripheral nerve regeneration applications.

Thus, in a further aspect, the present invention relates a wound closure material, suture material, soft tissue (skin) material, muscle material, connective tissue (tendon and/or ligament) material, cartilage material, bone material, or bridging aid in peripheral nerve regeneration comprising or consisting of the collagen and silk (collagen-silk) polypeptide composite structure as described herein.

Preferably, the biofabrication includes tissue, bone, tendon, or cartilage construction.

The use is specifically in vitro or ex vivo.

In a seventh aspect, the present invention relates to the (in vitro) use of a collagen solution and a silk polypeptide solution for producing/forming a collagen and silk (collagen-silk) polypeptide composite structure.

The special feature of this use is that neither the collagen solution nor the silk polypeptide solution comprises a separate cross-linker molecule. In other words, the collagen and the silk polypeptide are directly linked/associated with each other (without (the need of) a separate cross-linker molecule). The collagen and silk (collagen-silk) polypeptide composite structure also does not need to be re-treated with linker molecules to induce subsequent crosslinking. In the past, printed collagen or collagen composite structures were additionally treated using chemical cross-linking agents to obtain covalent bonds. Typical cross-linking strategies contained glutaraldehyde-(GA), 1-ethyl-3-(-3-dimethylaminopropyl) carbodiimide hydrochloride (EDC)/N-Hydroxysulfosuccinimide (NHS), genipin, Irgacure®, lithium-Phenyl-2,4,6-trimethylbenzoylphosphinate (LAP), ruthenium, formaldehyde (FA), epoxy-functional cross-linking agent, or combinations thereof. This is not necessary anymore. The avoidance of cross-linking has the great advantage that no (cell) toxic substances are part of the composite structure. This is very important for any medical application.

In one preferred embodiment,
the concentration of the collagen in the collagen solution is between 5 mg/ml and 20 mg/ml, more preferably between 10 mg/ml and 20 mg/ml, even more preferably between 12 mg/ml and 15 mg/ml, and still even more preferably 15 mg/ml, e.g. 5, 6, 7, 8, 9, 10, 11, 12, 13, 14, 15, 16, 17, 18, 19, or 20 mg/ml, and/or
the concentration of the silk polypeptide in the silk polypeptide solution is between 5 mg/ml and 40 mg/ml or between 5 mg/ml and 50 mg/ml, more preferably between 10 mg/ml and 30 mg/ml, even more preferably between 12 mg/ml and 25 mg/ml, and still even more preferably 20 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml.

In one also (alternatively or additionally) preferred embodiment,
the pH of the collagen solution is between pH 2.0 and 4.0, more preferably between pH 2.5 and 3.5, and even more preferably pH 3.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and/or
the pH of the silk polypeptide solution is between pH 6.5 and 8.5, more preferably between pH 7.0 and 7.8, and even more preferably pH 7.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

In one particularly preferred embodiment,
the concentration of the collagen in the collagen solution is between 5 mg/ml and 20 mg/ml, e.g. 5, 6, 7, 8, 9, 10, 11, 12, 13, 14, 15, 16, 17, 18, 19, or 20 mg/ml, and the pH of the collagen solution is between pH 2.0 and 4.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and/or
the concentration of the silk polypeptide in the silk polypeptide solution is between 5 mg/ml and 40 mg/ml or between 5 mg/ml and 50 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml, and the pH of the silk polypeptide solution is between pH 6.5 and 8.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5.

The collagen solution and/or the silk polypeptide solution may further comprise an additive matter, i.e. one or more additive molecules. In this way, the functionality of the collagen and silk (collagen-silk) polypeptide composite structure can be increased.

In one further (alternatively or additionally) preferred embodiment,
the additive matter is comprised in the collagen solution in an amount in the range of 0.01 wt %/vol to 10 wt %/vol, and more preferably in an amount in the range of 0.5 wt %/vol to 1 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol, and/or
the additive matter is comprised in the silk polypeptide solution in an amount in the range of 0.01 wt %/vol to 10 wt %/vol, and more preferably in an amount in the range of 0.5 wt %/vol to 1 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

In one further particularly preferred embodiment,
the concentration of the collagen in the collagen solution is between 5 mg/ml and 20 mg/ml, e.g. 5, 6, 7, 8, 9, 10, 11, 12, 13, 14, 15, 16, 17, 18, 19, or 20 mg/ml, the pH of the collagen solution is between pH 2.0 and 4.0, e.g. pH 2.0, 2.1, 2.2, 2.3, 2.4, 2.5, 2.6, 2.7, 2.8, 2.9, 3.0, 3.1, 3.2, 3.3, 3.4, 3.5, 3.6, 3.7, 3.8, 3.9, or 4.0, and the collagen solution comprises an additive matter in an amount in the range of 0.01 wt %/vol to 10 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol, and/or
the concentration of the silk polypeptide in the silk polypeptide solution is between 5 mg/ml and 40 mg/ml or between 5 mg/ml and 50 mg/ml, e.g. 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 mg/ml, the pH of the silk polypeptide solution is between pH 6.5 and 8.5, e.g. pH 6.5, 6.6, 6.7, 6.8, 6.9, 7.0, 7.1, 7.2, 7.3, 7.4, 7.5, 7.6, 7.7, 7.8, 7.9, 8.0, 8.1, 8.2, 8.3, 8.4, or 8.5, and the silk polypeptide solution comprises an additive matter in an amount in the range of 0.01 wt %/vol to 10 wt %/vol, e.g. 0.01, 0.05, 0.1, 0.2, 0.3, 0.4, 0.5, 0.6, 0.7, 0.8, 0.9, 1.0, 1.5, 2.0, 2.5, 3.0, 3.5, 4.0, 4.5, 5.0, 5.5, 6.0, 6.5, 7.0, 7.5, 8.0, 8.5, 9.0, 9.5, or 10.0 wt %/vol.

The collagen solution and/or the silk polypeptide solution may further comprise an additive matter, e.g. one or more additive compounds. It does not matter whether the collagen solution or the silk polypeptide solution, or both comprise the additive matter. In case of the collagen solution, the additive matter is preferably selected from the group consisting of beads, electricity conducting metals, crystallites, and biologically active agents, preferably pharmaceutical active compounds. In case of the silk polypeptide solution, the additive matter is preferably selected from the group consisting of cells, beads, electricity conducting metals, crystallites, and biologically active agents, preferably pharmaceutical active compounds.

The above-mentioned collagen solution is preferably an aqueous solution, e.g. water or an aqueous buffer comprising collagen, and/or the above-mentioned silk polypeptide solution is preferably an aqueous solution, e.g. water or an aqueous buffer comprising silk polypeptides. More preferably, the collagen solution is an acidic collagen solution and the silk polypeptide solution has a physiological pH (a pH of about 7.5). Even more preferably, the collagen solution has a pH as indicated in the first aspect of the present invention and the silk polypeptide solution has a pH as indicated in the second aspect of the present invention.

In an eighth aspect, the present invention relates to a system for carrying out the method according to the first aspect, wherein the system comprises a processor capable of executing machine readable instructions when the machine-readable instructions are loaded into the system.

In a ninth aspect, the present invention relates to a computer program comprising machine-readable instructions which when loaded into the system according to the eight aspect cause the system to carry out the method according to the first aspect.

Various modifications and variations of the invention will be apparent to those skilled in the art without departing from the scope of invention. Although the invention has been described in connection with specific preferred embodiments, it should be understood that the invention as claimed should not be unduly limited to such specific embodiments. Indeed, various modifications of the described modes for carrying out the invention which are obvious to those skilled in the art in the relevant fields are intended to be covered by the present invention.

### BRIEF DESCRIPTION OF THE FIGURES

The following figures are merely illustrative of the present invention and should not be construed to limit the scope of the invention as indicated by the appended claims in any way.

FIG. 1: Shows a 3D printing process of collagen I/III into an eADF4(C₁₆) precipitation bath. Photographs of the constructs (A) during the printing process, (B) after incubation in eADF4(C₁₆) solution, and (C) after the second incubation in post treatment buffer. (D) SEM image shows the structure of the constructs consisting of collagen fibrils and eADF4(C₁₆) particles.

FIG. 2: Shows a representative fitted stress-compression diagrams of collagen-eADF4(C₁₆) (black) and collagen-eADF4(C₁₆-RGD) (light grey) scaffolds. The collagen-eADF4(C₁₆-RGD) scaffolds show a higher compressive stress than the collagen eADF4(C₁₆) scaffolds.

FIG. 3: Cumulative enzymatic protein degradation of collagen-eADF4(C₁₆) (light grey) and pure collagen (gray) scaffolds in protease mixture PXIV (square), collagenase mixture CHC (circle), and buffer control TCNB (triangle) over 7 days and macroscopic images of the samples after 1, 4 and 7 days. The protein degradation was normalized to PXIV coll-eADF4(C₁₆) samples.

FIG. 4: Development of a skin model. Fully developed fibroblast layer (BJ Morph) on the (A) collagen-eADF4(C₁₆), (B) collagen-eADF4(C₁₆-RGD), and (C) pure collagen-scaffolds. Co-culture of the greenlabeled keratinocytes (Syto™ HaCaT) on the fibroblast layer were only possible on the (D) collagen-eADF4(C₁₆) and (E) collagen-eADF4(C₁₆-RGD) scaffolds due to higher stability compared to pure collagen scaffolds.

FIG. 5: Confocal microscopy images of encapsulated fibroblasts within the 3D-printed collagene ADF4(C₁₆-RGD)- and collagen-constructs after 1 (BJ morph, expressed tomato red protein) and 17 (calcein, live staining) days in cell culture.

FIG. 6: Examination of collagen filaments printed in 20 mg/ml unlabeled (A and D), 10% (B and E), and 100% (C and F) rhodamine-labeled eADF4(C₁₆) solution. (G) SEM of the collagen-silk-composite showing the highly repetitive D-band pattern (black arrows indicate the 67 nm band) typical for native collagen fibrils and spider silk particles.

FIG. 7: Effect of the eADF4(C₁₆) bath concentration, namely (A) 20 mg/ml, (B) 30 mg/ml, (C)₄₀ mg/ml and (D) 50 mg/ml eADF4(C₁₆) in Tris buffer (pH 7.5), on the collagen-silk-scaffold formation after 3D-printing and addition of 1 M potassium phosphate (KPi).

FIG. 8: Turbidity measurements at 340 nm in relative absorbance units (RAU) for a time period of 300 min to analyse collagen self-assembly behavior in Tris buffer (blue), eADF4(C₁₆) (black) and eADF4(C₁₆-RGD) (green) solution upon addition (A) and absence (B) of 1 M KPi buffer (pH 8.0) after 120 min indicated by brown arrows. SEM analysis of assembled collagen and Coll-silk structures in presence (C) and absence (D) of KPi buffer. The addition of 1 M KPi after 120 min resulted in particle formation of the spider silk proteins (I+II). White stars designate additional collagen aggregation upon KPi addition (III). In contrast, in the absence of KPi, collagen assembles into regular fibrils (VI), and spider silk also self-assembles over time leading to entanglements and interconnections indicated by white arrows (IV+V). (E) Schematic illustration of the collagen and silk assembly during incubation in Tris buffer (pH 7.5) before and after protein precipitation using 1 M KPi buffer (pH 8.0).

FIG. 9: Schematic representation of cell culture experiments to fabricate a two-layer skin model consisting of human skin fibroblasts (BJ Morph, red) and keratinocytes (HaCaT, SYTO-dyed, green) seeded on 3D-printed collagen-spider silk composite scaffolds. Representative confocal microscopy images of fibroblasts after (A) seeding, (B) 14 days of cultivation and (C) co-cultivation using keratinocytes after 36 days.

FIG. 10: SEM analysis of BJ morph fibroblasts cultured on Coll-eADF4(C₁₆), Coll-eADF4(C₁₆-RGD) and Coll-Tris scaffolds after 1, 7 and 14 days.

### EXAMPLES

The examples given below are for illustrative purposes only and do not limit the invention described above in any way.

### INTRODUCTION

Collagen is one main component of the extracellular matrix (ECM) in natural tissues and is, therefore, well suited as a biomaterial for tissue engineering such as skin tissue engineering. However, it has proven difficult to produce collagen scaffolds that can replicate the structure and function of tissues and organs. In this experimental section, 3D printing of a concentrated collagen solution (e.g. 15 mg/ml) into a pH-neutral aqueous precipitation bath of recombinant spider silk eADF4(C₁₆) (e.g. 20 mg/ml) with or without the RGD motif (e.g. in Tris buffer, pH 7.5) was performed to overcome the limitations mentioned above. Collagen fibril formation of an acidic collagen I/III solution occurred upon contact with the spider silk precipitation bath due to pH increase, e.g. from 3.0 to 7.5. This precipitation occurred in a suitable time range that allowed a layer-by-layer deposition of the collagen ink in order to form a stable 3D construct with high shape fidelity. Subsequent addition of a phosphate containing buffer (e.g. 1 M potassium phosphate (KPi, pH 8.0)) induced spider silk particle formation, strengthened fibril formation, and resulted in mechanically stable particle-reinforced scaffolds. Using the RGD-containing spider silk variant within the precipitation bath resulted in a scaffold showing superior mechanical properties and additional cell interaction sites compared to ones using the unmodified eADF4(C₁₆) silk variant. The resulting collagen-silk composite scaffolds are biocompatible, biodegradable, bioactive, and promising for tissue engineering applications. All experimental details are described in the following sections:

### Example 1

### 1. 3D Printing Process

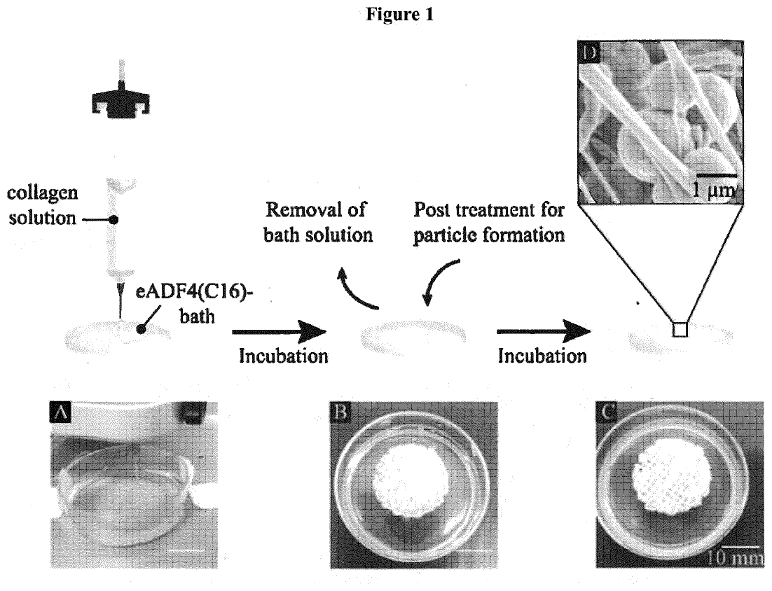

In the 3D printing process, a 15 mg/ml collagen solution was extruded into a precipitation bath composed of aqueous eADF4(C₁₆ or C₁₆-RGD) solution at a concentration of 20 mg/ml at pH 7.5. The precipitation bath allowed the continuous layer-by-layer printing of the collagen solution with a solid and uniform bonding of the individual layers. After printing, the constructs were left in the silk solution for an incubation time of 2 h at room temperature. This resulted in complete cross-linking and fibril formation of the collagen filaments, which could be recognized by the colour change (from transparent to white). Subsequently, the silk solution was removed, and the constructs were incubated in 1M potassium phosphate buffer at 37° C. overnight (only without cells, shorter for cell-containing samples) to induce the formation of silk particles from unstructured silk proteins (see FIG. 1, flow chart). In FIGS. 1A-C, photographs of the constructs are shown. FIG. 1A shows the construct during the printing process. FIG. 1B shows the construct after incubation in eADF4(C₁₆) solution. FIG. 1C shows the construct after the second incubation in post treatment buffer. This construct consists of collagen fibrils and eADF4(C₁₆) particles. FIG. 1D shows a SEM image which confirmed the existence of collagen fibrils and subsequent silk particle formation, visualized using fluorescence microscopy.

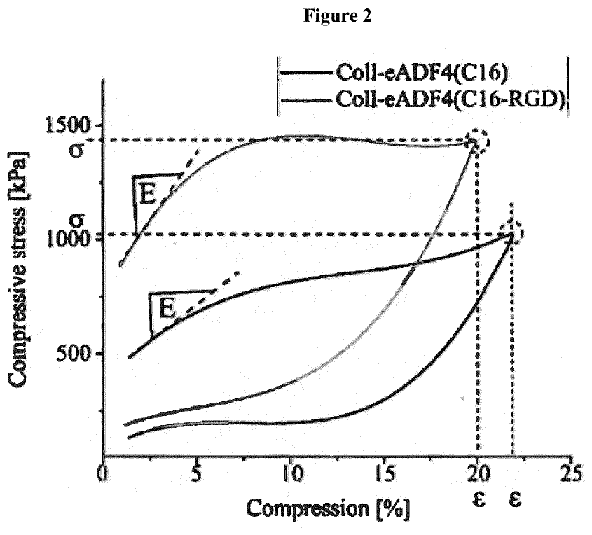

Besides unmodified eADF4(C₁₆), a variant containing an RGD motif can also be used within the precipitation bath resulting in superior mechanical properties compared to the unmodified silk variant (see FIG. 2).

### 2. Stability of the Printed Collagen-Silk Composite Structure

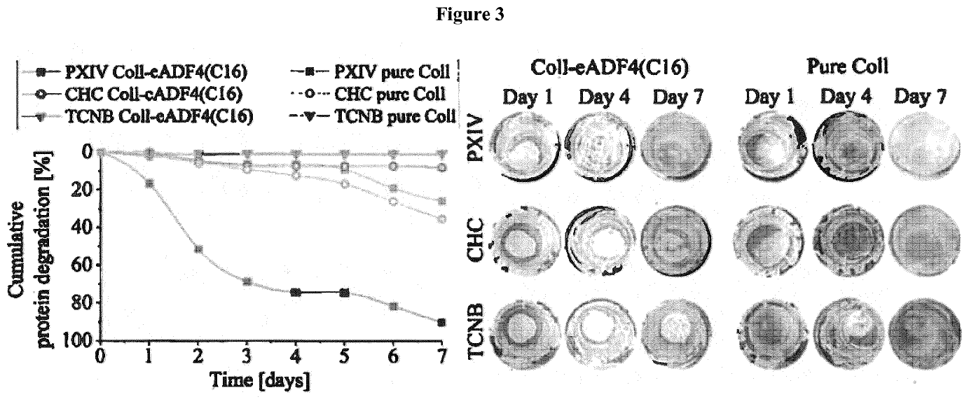

To evaluate the stability of the 3D-printed composite scaffolds at wound-like or digestive conditions, in vitro enzymatic degradation was performed over seven days. Besides the superior mechanical properties of the collagen-silk scaffolds, the combination of these two structural proteins leads to a slower degradation rate of the scaffolds at wound conditions compared to collagen scaffolds alone (see FIG. 3). FIG. 3 shows cumulative enzymatic protein degradation of collagen-eADF4(C₁₆) (light grey) and pure collagen (grey) scaffolds in protease mixture PXIV (square), collagenase mixture CHC (circle), and buffer control TCNB (triangle) over 7 days and macroscopic images of the samples after 1, 4 and 7 days. The protein degradation was normalized to PXIV coll-eADF4(C₁₆) samples.

Specifically, the degradation of pure Coll samples in this wound condition model showed greater degradation, whereas the Coll-eADF4(C₁₆) samples were more stable and showed a slow degradation. Thus, spider silk particles seemed to hinder fast collagen degradation at these conditions, which is important to allow cells to attach and built up new extracellular matrix and regenerate tissue during wound healing.

### 3. Development of a Collagen-Silk Composite Structure Skin Model

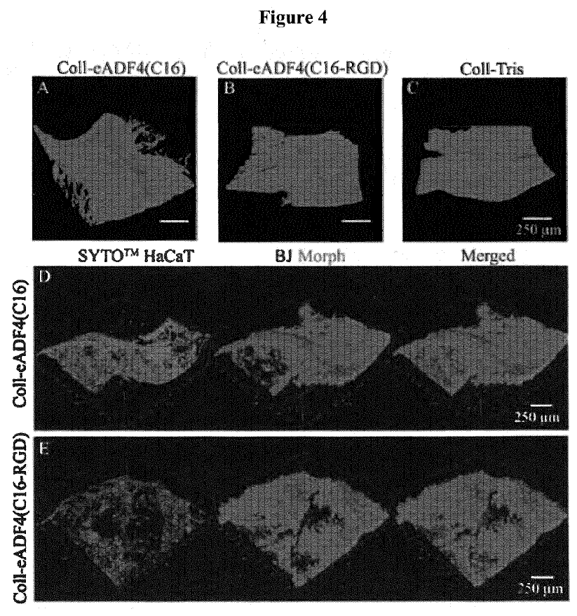

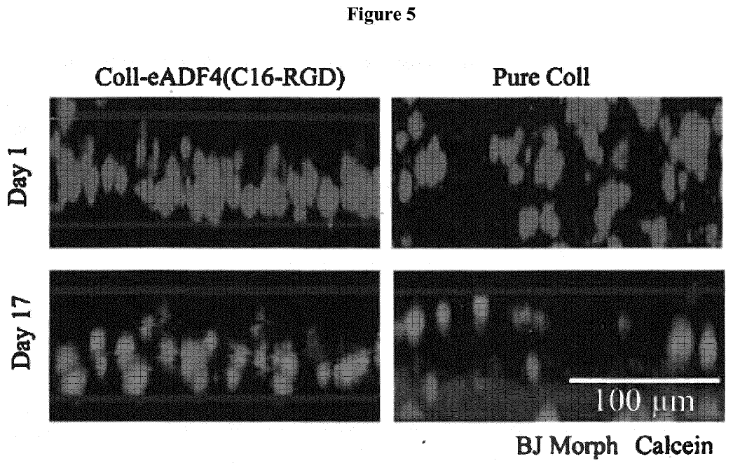

3D printed scaffolds were further seeded with BJ morph fibroblasts and kept in culture for 14 days. Afterward, the collagen-silk- and pure collagen-scaffolds were completely covered with fibroblasts (see FIG. 4A-C). The cells were cultured for additional 7 days before co-culturing with keratinocytes (HaCaTs) to develop a full-length skin model. For this purpose, HaCaTs were labeled using SYTO green fluorescent dye and seeded onto the fibroblast layer of the collagen-silk scaffolds. The collagen-eADF4(C₁₆) and collagen-eADF4(C₁₆-RGD) scaffolds showed a complete BJ and HaCaT layer after 8 days of culture (see FIG. 4D-E). Due to the loose and fibrous structure of the pure collagen constructs printed in Tris buffer, no co-cultivation could be performed. The physiological conditions of the eADF4(C₁₆) and eADF4(C₁₆-RGD) precipitation bath (pH 7.5 and appropriate cell culture media), allow the implementation of cells, which are homogenously integrated into the scaffolds during the 3D printing process. Subsequent addition of 1 M potassium phosphate buffer induces particle formation, strengthens fibril formation, and results in mechanically stable scaffolds without cell harming. The cells integrated into the 3D printed structure were also viable up to 17 days in culture (see FIG. 5).

### 4. Disadvantages of the Prior Art and Advantages of the Present Invention

The disadvantages of the prior art are summarized as follows:
poor printability of collagen solutions/inks
poor mechanical properties of pure collagen scaffolds, which require crosslinking of 3D-printed constructs using toxic substances in order to obtain mechanically stable constructs
particle reinforcement of nano-fibrillar structures was only carried out using inorganic fillers.

The advantages of the present invention are summarized as follows:
extrusion/printing of a mechanically stable collagen and silk (collagen-silk) polypeptide composite structure without the need for toxic cross-linking methods
the method has a high printing resolution and shape fidelity
the collagen and silk (collagen-silk) polypeptide composite structure which is obtained with the above method is biodegradable and biocompatible.
further improved mechanical properties by using the RGD modified silk polypeptide variant eADF4(C₁₆-RGD),
particle reinforcement of a fibrillary system by fully protein-based structures leading to high biocompatibility and cell adhesiveness
encapsulation of cells inside the construct is possible by adding the cells to the silk polypeptide precipitation bath
controllable, slow biodegradation to allow generation of cell-made ECM before degradation of supporting matrix

### Example 2

### 1. Experimental Section

### Materials

Collagen I/III grist from equine deep flexor tendon was provided by RESORBA (Nuremberg, Germany). Recombinant eADF4(C₁₆) spider silk powder was purchased from AMSilk GmbH (Munich, Germany). Dulbecco's Modified Eagle's Medium (DMEM) and fetal calf serum (FCS) were purchased from BioSell (Germany). Eagle's minimum essential medium (EMEM) and gentamycin sulfate were purchased from Sigma-Aldrich (Germany). Ethanol, hydrochloric acid (HCl), and phosphate buffered saline (PBS) were obtained from VWR (Darmstadt, Germany). 4′,6-diamidino-2-phenylindole dihydrochloride (DAPI), the focal adhesion staining kit, GlutMax (Gibco), dialysis membranes (Spectra/Por), and treated, as well as non-adherent well plates were obtained from Thermo Fisher Scientific (Bonn, Germany). All other chemicals were purchased from Carl-Roth GmbH+Co KG (Karlsruhe, Germany).

### Preparation of Printing Solutions

Collagen I/III grist was dissolved in 10 mM HCl at a concentration of 15 mg/ml to achieve homogeneous solutions. The mixture was shaken vigorously until no pieces were visible, and mixing was continued for two days at 4° C. Next, it was filtered through a membrane (90×90 μm mesh size) and centrifuged at 10,000 g for 10 min to remove air bubbles.

Recombinant eADF4(C₁₆)-RGD spider silk protein was recombinantly produced and purified as described previously. The freeze-dried spider silk proteins were dissolved in 6 M guanidinium thiocyanate at concentrations between 10 and 20 mg/ml and dialyzed against 10 mM Tris/HCl buffer (pH 7.5) using membranes with a molecular weight cutoff of 6-8 kDa. Afterwards, spider silk solutions were dialyzed against 25% (w/v) polyethylene glycol to increase the protein concentration as previously described. The protein solutions were centrifuged (13,300 rpm, 10 min) to remove protein aggregates. For the analysis of the scaffold microstructure, eADF4(C₁₆) was modified with rhodamine using a chemical covalent coupling as described previously.

### 3D-Printing and Scaffold Fabrication

Collagen solutions were extruded into a coagulation bath consisting of aqueous spider silk solutions at concentrations of 20, 30, 40 and 50 mg/ml using a 3D Discovery printer (regenHU, Villaz-St-Pierre, Swiss) with a feed rate of 5 mm/s. Pneumatic print heads were equipped with 3 cc cartridge, pistons, and a 25 G steel needle with an inner diameter of 260 μm (drifton, Denmark). The printing pressure was adjusted manually for optimal printing results (0.8-1.4 bar). Collagen solution printing in 10 mM Tris/HCl-buffer was used as a control. Printed constructs were incubated for 2 h in coagulation baths (spider silk solutions and Tris-buffer) at RT. Afterwards the bath solution was removed and exchanged using 1 M KPi (potassium phosphate) (pH 7.5). The scaffolds were incubated overnight in a cell culture incubator (95% humidity) at 37° C.

### Analysis of the Scaffold Microstructure

The microstructure of the individual protein components and their interaction were studied using a rhodamine-labeled eADF4(C₁₆) solution as a precipitation bath. For this purpose, 15 mg/ml collagen solution was printed into a precipitation bath (20 mg/ml) containing 100% (v/v) unlabeled, 10% (v/v) Rhodamine-labeled and 100% (v/v) Rhodamine-labeled eADF4(C₁₆). Samples were analyzed afterwards using light and fluorescence microscopy, as well as scanning electron microscopy (SEM, Carl Zeiss Microscopy GmbH, Germany and Apreo VS, Thermo Fisher Scientific, Germany). For the latter, the samples were dehydrated using a gradual concentration of ethanol (50, 70, 80, 90, 100% ethanol in water). Each step was conducted for 30 min, except the last step that was conducted over night until ethanol was completely evaporated. Afterwards, samples were treated with pure tert-butanol for 5 min at RT. Samples were frozen at −80° C. for 1 h before lyophilization overnight. Before imaging using SEM, samples were sputter-coated for 30 s at 30 mA with 2 nm platinum and imaged subsequently.

### Assembly Kinetics

To simulate the collagen-spider silk scaffold fabrication process, 100 μl of collagen solution was added to a 96-well plate, and 100 μl of eADF4(C₁₆) (pH 7.5), eADF4(C₁₆-RGD) (pH 7.5), or Tris buffer (pH 7.5) each, was added. The turbidity was measured at 570 nm. After 2 h, the turbidity measurement was interrupted, 1 M KPi buffer was added per 100 μl, and the measurement was continued for 16 h using an automated plate reader system (SpectraMax iD5, Molecular Devices).

### Cell Culture Experiments

Human BJ Morph fibroblasts were kindly provided by Lena Fischer and Dr. Ingo Thievessen (Biphysics group, University Erlangen-Nuremburg, Germany). BJ Morph fibroblasts were cultured in Eagle's minimum essential medium (EMEM) supplemented with 10% (v/v) fetal calf serum (FCS), 1% (v/v) GlutaMax and 0.1% (v/v) gentamycin sulphate. Human HaCaT keratinocytes (DKFZ, CLS, Germany) were cultured in DMEM supplemented with 10% (v/v) FCS, 1% (v/v) GlutaMax and 0.1% (v/v) gentamycin sulphate. Both cell lines were cultured in a cell culture incubator (HeraCell, Thermo Fisher Scientific) at humidified conditions containing 5% CO₂ at 37° C. Subculturing was conducted using trypsin/EDTA. Cell numbers and viability were evaluated using trypan blue and an automated cell counter (TC20, Bio-Rad Laboratories, Germany). To analyse the cytocompatibility and adhesion behavior on 3D printed constructs, 60,000 BJ Morph fibroblasts were seeded onto each scaffold. The samples were incubated over 14 days and analysed regarding cell proliferation.

For co-culture, HaCaT keratinocytes were stained using Syto 9 DNA stain (green, Invitrogen, Thermo Fisher Scientific) to allow a specific identification of keratinocytes (green) and BJ Morph (red) using fluorescence microscopy. Therefore, HaCaT keratinocytes were incubated with 1 μM Syto 9 DNA stain in DMEM for 1 h at 37° C. Afterwards, the cells were washed several times using 1×PBS to remove unspecifically surface bound dye. Keratinocytes were resuspended in DMEM/EMEM (1:1), and 50,000 cells were seeded on each scaffold (Coll-Tris, Coll-eADF4(C₁₆), Coll-eADF4(C₁₆)-RGD) containing confluent BJ-layers (cultured for 21 days).

### Analysis of Cell Proliferation, Morphology and Viability

Cell proliferation of BJ Morph fibroblasts and Syto 9-stained HaCaT keratinocytes on 3D-printed constructs were analyzed using a Leica DMI 8 confocal laser scanning microscope and the associated LAS X software (both Leica, Germany). For focal adhesion staining, the samples were fixed using 4% (v/v) paraformaldehyde in 1×PBS and permeabilized using 0.1% (v/v) triton-X in 1×PBS. Samples were blocked using BSA solution.

### 2. Results

### Analysis of the Effect of Spider Silk Proteins on Collagen I/III Assembly and Precipitation

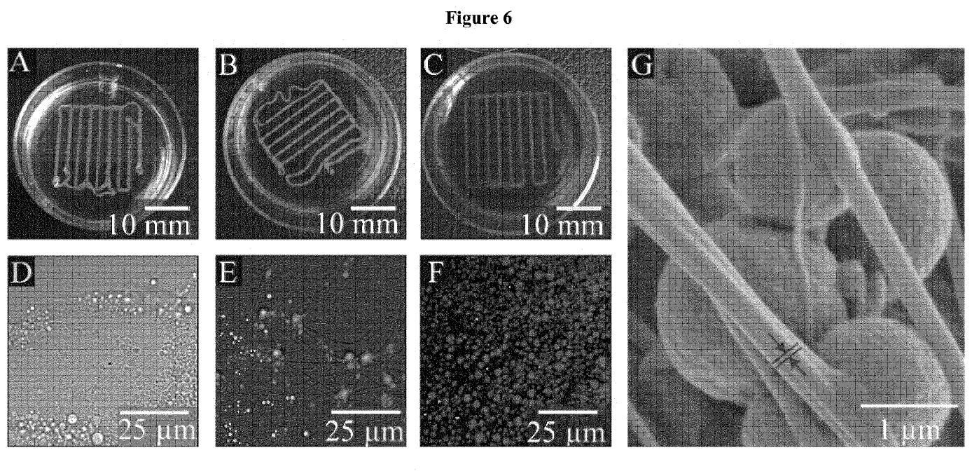

To analyze the effect of the spider silk proteins on collagen I/III assembly and precipitation in more detail, the collagen solution was manually extruded in 10 mg/ml aqueous recombinant spider silk solution made of eADF4(C₁₆) or Tris buffer (pH 7.5) without protein. Compared to Tris buffer, the spider silk protein solution supported collagen precipitation indicated by a faster turbidity change from transparent to a whitish color. Furthermore, the subsequent addition of KPi led to spider silk particle formation and stabilization of the extruded collagen indicated by mechanically stable, white strands (data not shown). A single-layer square with 20×20 mm and a strut spacing of 1.35 mm was printed into a precipitation bath consisting of rhodamine-labeled eADF4(C₁₆) solutions to investigate, if spider silk is only assembled in particles or also incorporated in the collagen fibrils. Therefore, 15 mg/ml collagen solution was printed into spider silk precipitation baths (20 mg/ml) either containing 100% (v/v) unlabeled (FIG. 6, A), 90% (v/v) unlabeled and 10% (v/v) Rhodamine-labeled (FIG. 6, B) or 100% (v/v) Rhodamine-labeled (FIG. 6, C) eADF4(C₁₆). Using light and fluorescence microscopy, the two different morphologies of the proteins, namely fibrils and particles, could be visualized (FIG. 6, D-F). The fluorescently labeled samples showed red eADF4(C₁₆) particles and unlabeled collagen fibrils connecting the silk particles (FIG. 6, E and F). Thus, the increase of pH led to collagen fibril formation, while spider silk particles were precipitated using the potassium phosphate. The particle-reinforced, fibrillar microstructure of the filaments was further analyzed using SEM after dehydration (FIG. 6, G).

### Determination of the Optional Precipitation Bath Concentration

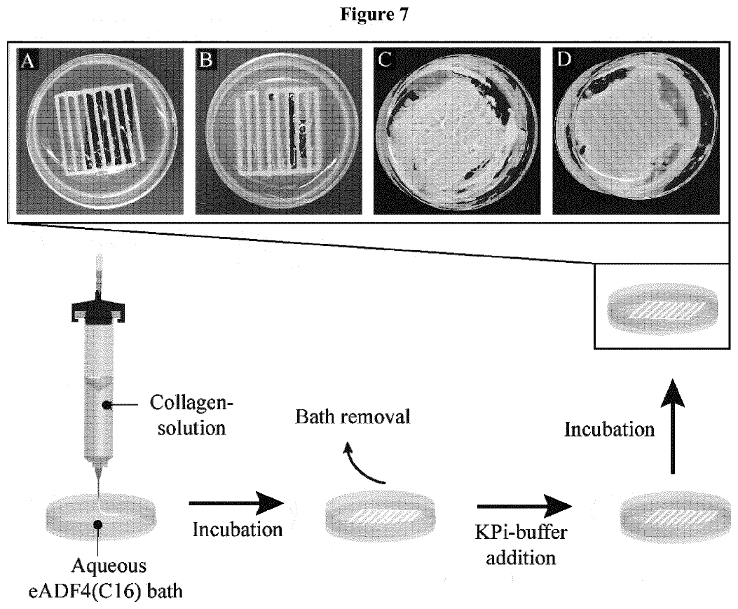

To determine the optimal precipitation bath concentration, the 15 mg/ml collagen solution was printed into 20, 30, 40 and 50 mg/ml eADF4(C₁₆) solutions (FIG. 7). As a control, collagen was printed into pure Tris buffer (pH 7.5, data not shown). Printing of the collagen solution in eADF4(C₁₆) precipitation baths showed uniform filaments at all concentrations. The resulting filaments precipitated slowly in the eADF4(C₁₆) solutions, changing the color of the collagen filament within 40-60 s from transparent to turbid. After incubation in the silk solutions and subsequent precipitation of spider silk particles using KPi, clear demarcations between the collagen filaments were visible at eADF4(C₁₆) concentrations of 20 and 30 mg/ml (FIG. 7, A and B). At higher spider silk concentrations (40 and 50 mg/ml), the eADF4(C₁₆) solution settled around the collagen filaments like a film (FIG. 7, C and D). In any case, with increasing protein concentration, the fibril formation accelerated.

### Development of a Self-Assembly Model of Collagen Fibrils and Spider Silk Particles

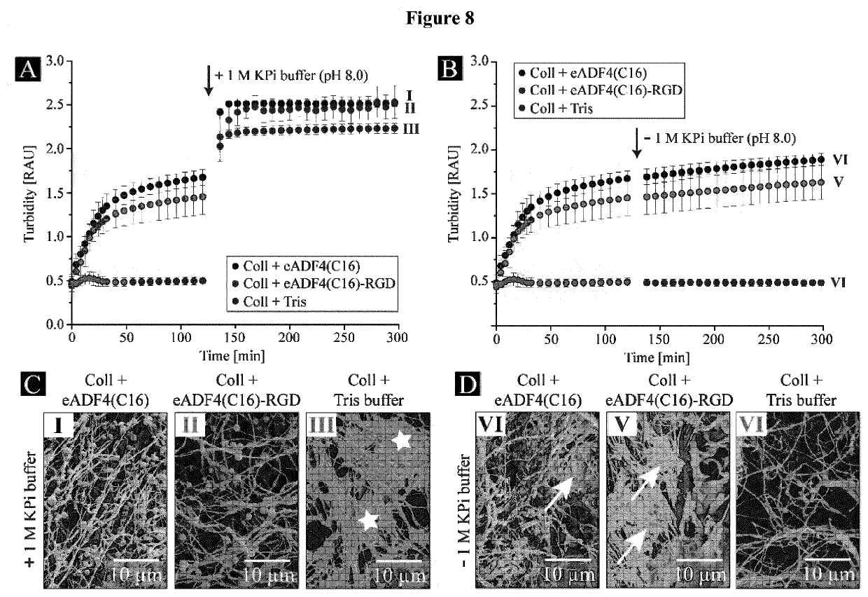

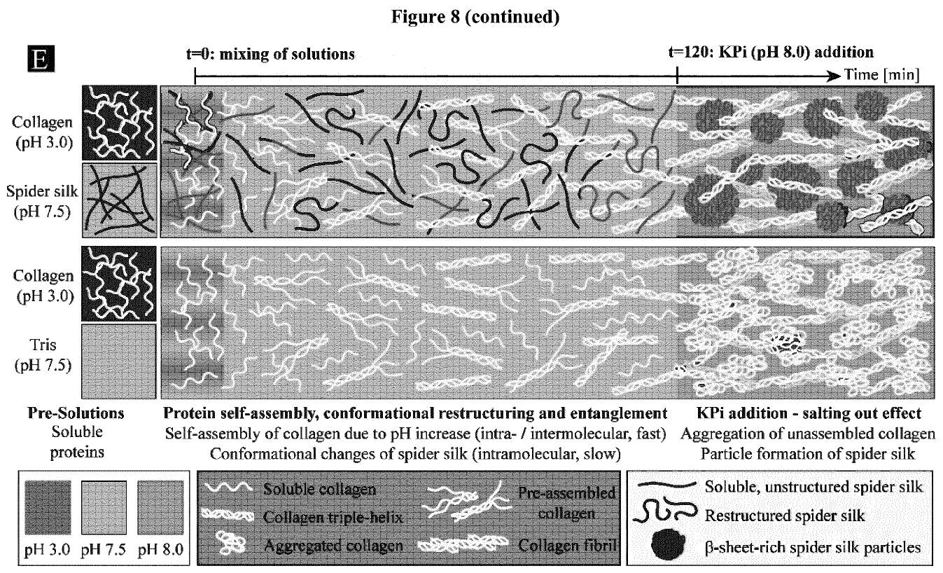

In order to develop a self-assembly model of collagen fibrils and spider silk particles, time-dependent turbidity measurements and SEM measurements of the resulting scaffolds were conducted (FIG. 8). In this context, eADF4(C₁₆)-RGD was used with regard to the tissue engineering application of the collagen-silk-scaffolds. The turbidity measurements showed that self-assembly and fibril formation of collagen started immediately after the addition of the spider silk solutions or Tris buffer (FIG. 8, A+B). The turbidity of the Coll-Tris samples was more or less similar during the 120 min incubation time indicating a slow self-assembly into collagen triple-helices without twists and entanglements. In contrast, turbidities induced by eADF4(C₁₆) or eADF4(C₁₆-RGD) solutions continuously increased up to minute 120 indicating beneficial, additional conformational changes, restructuring and entanglements induced by silk molecules. The addition of KPi (pH 8.0) increased the turbidity of all three samples significantly (FIG. 8, A). These similar curve progressions indicated the precipitation of unsassembled collagen and spider silk molecules. However, the precipitation of recombinant spider silk particles increased the apparent turbidity further. In contrast, samples incubated without KPi displayed an even curve progression and constant increase in turbidity indicating continuous protein self-assembly (FIG. 8, B).

Afterwards, the samples were gradually dehydrated through an alcohol series, freeze-dried, and the morphology was examined using SEM (FIG. 8, C+D). FIG. 8 D shows the samples without additional KPi. Collagen fibrils were visible in all samples. The diameters of the collagen fibrils were around 200 nm within the SEM images determined using Image J software. However, additional eADF4(C₁₆)- and eADF4(C₁₆-RGD)-assemblies were detectable between the collagen fibrils (FIG. 8 D, white arrows) leading to twists, entanglements and interconnections responsible for the turbitiy increase of spider silk-containing samples. The addition of KPi resulted in the formation of eADF4(C₁₆) and eADF4(C₁₆-RGD) particles by salting out (FIG. 8 C). The diameter of the resulting spider silk particles ranged around 1.3 μm. In general, collagen was able to self-assemble in nanofibrils in Tris buffer in presence and absence of KPi. However, the self-assembly was not finished after 120 min leading to increased aggregation of unassembled collagen after addition of KPi. In addition to single collagen fibrils, many aggregated, melted sheets were visible (FIG. 8 C, white stars) explaining the significant raise of turbidity.

Based on the turbidity and SEM results, collagen assembly was sketched in absence and presence of spider silk in Tris buffer (pH 7.5) and upon addition of KPi buffer. The resulting structures are influenced by the pH-driven collagen assembly as well as the conformational restructuring and entanglement of collagen and spider silk (FIG. 8 E).

### Fabrication of a Two-Layer Skin Model

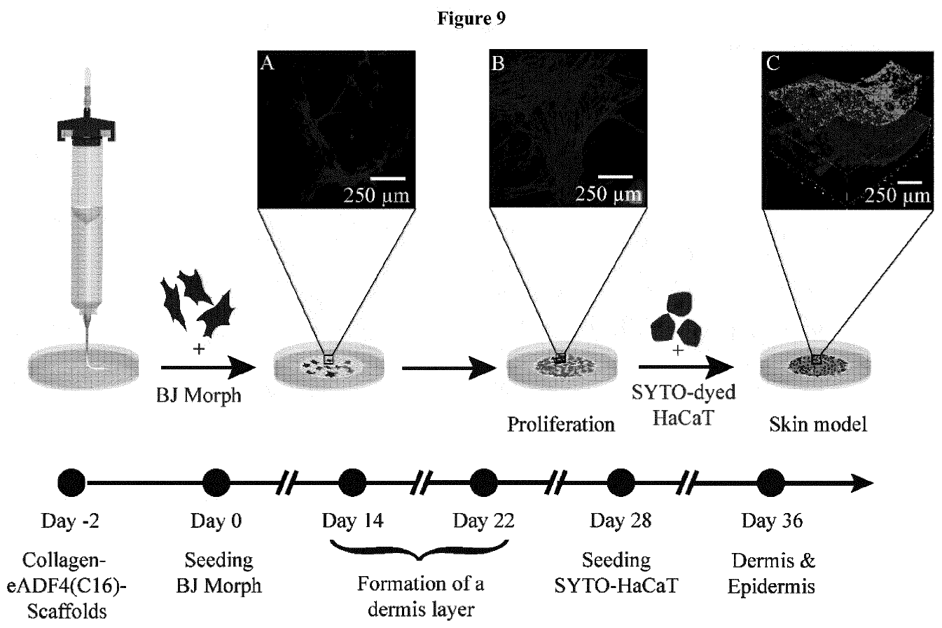

The cells were cultured for 28 days followed by a co-culture with human skin keratinocytes (HaCaTs) to develop a two-layer skin model (FIG. 9). HaCaT keratinocytes were labeled with SYTO green fluorescent nucleic acid stain, seeded onto the fibroblast layer of the collagen-spider silk scaffolds, and cultured for additional 8 days (36 days in total). The coll-eADF4(C₁₆) and coll-eADF4(C₁₆-RGD) scaffolds showed complete BJ Morph (red) and HaCaT (green) layers after 8 days of culture (FIG. 9, C). Cell culture experiments demonstrated that the collagen-spider silk composite scaffolds provided a good environment for skin cells (fibroblasts, keratinocytes) and, thus, will enable skin tissue engineering in the future.

Interaction of Cells with the Surrounding Scaffold

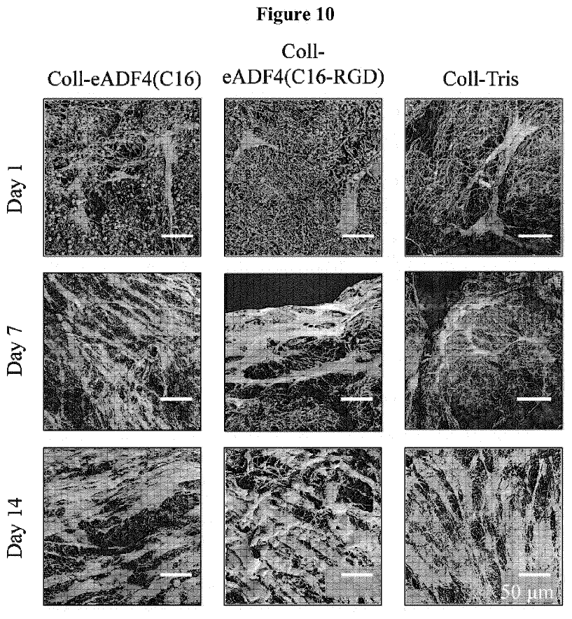

The interaction of cells with the surrounding scaffold was further considered using SEM to analyses cell interdependency with scaffold topography and possible cellular anchoring points (FIG. 10). SEM analysis indicated that fibroblasts not only adhered to the scaffold surfaces but also migrated into the interstices of the fibrils. Cells were located both above and below the collagen fibrils and spider silk particles. This was particularly evident with the lower cell number on day 1 (FIG. 10). In addition, the cells spread along the collagen fibrils. Significantly larger cell numbers were observed on day 7 and 14. The cell culture results indicated that cell adhesion was mainly influenced by biochemical and topographical stimulation of the collagen. Since cell growth was comparable on Coll-eADF4(C₁₆) and Coll-eADF4(C₁₆)-RGD scaffolds, additional RGD cell interaction sites on the particles were apparently not mandatory for cell interaction.

### Summary

The combination of collagen and silk polypeptides in one 3D printing process is a promising approach for scaffold fabrication to be used in tissue regeneration. The fabricated scaffolds provide good growth conditions, since mechanical properties and slow biodegradation at wound-like conditions ensure cells the necessary stability during proliferation and tissue regeneration. The presented processing route combines the specific properties of the structural proteins collagen and silk in such a way that the unique functional, structural, and mechanical characteristics of the individual biopolymers enable the formation of scaffolds with desired characteristics. The resulting collagen-silk composite scaffolds are biocompatible. The silk particle reinforcement leads to enhanced mechanical properties and proteolytic stability of the collagen scaffolds.

1. A method for producing a collagen and silk polypeptide composite structure comprising the step of:
introducing a collagen solution into a silk polypeptide solution (precipitation solution),
thereby obtaining the collagen and silk polypeptide composite structure.

2. (canceled)

3. The method of claim 1, wherein the collagen and the silk polypeptide are directly linked/associated with each other (without (the need of) a separate cross-linker molecule).

4.-5. (canceled)

6. The method of claim 1, wherein
the pH of the collagen solution is between pH 2.0 and 4.0, and/or
the pH of the silk polypeptide solution is between pH 6.5 and 8.5.

7. (canceled)

8. The method of claim 1, wherein the collagen solution further comprises an additive matter, wherein the additive matter is comprised in the collagen solution in an amount in the range of 0.01 wt %/vol to 10 wt %/vol, and wherein the additive matter is selected from the group consisting of beads, electricity conducting metals, crystallites, and biologically active agents.

9.-10. (canceled)

11. The method of claim 1, wherein the silk polypeptide solution further comprises an additive matter, wherein the additive matter is comprised in the silk polypeptide solution in an amount in the range of 0.01 wt %/vol to 10 wt %/vol, and wherein the additive matter is selected from the group consisting of cells, beads, electricity conducting metals, crystallites, and biologically active agents.

12.-13. (canceled)

14. The method of claim 1, wherein the collagen solution is introduced layer-by-layer into the silk polypeptide solution.

15. The method of claim 1, wherein the collagen solution is introduced into the silk polypeptide solution via a (an extrusion) nozzle (which is part of a printing/extrusion unit), and wherein the silk polypeptide solution is comprised in a container.

16. (canceled)

17. The method of claim 15, wherein the (extrusion) nozzle (which is part of a printing/extrusion unit) and/or the container are movable relative to each other in a predetermined pattern along the x-, y- and z-axis to form the collagen and silk polypeptide composite structure, particularly each layer of said structure.

18. The method of claim 1, wherein the method comprises an incubation step for an incubation period, wherein during the incubation period no further collagen solution is introduced into the silk polypeptide solution.

19. (canceled)

20. The method of claim 18, wherein the incubation period is for between 10 min and 5 hours.

21.-24. (canceled)

25. The method of claim 18, wherein after expiration of the incubation period, the collagen and silk polypeptide composite structure is exposed to a buffer solution (post-treatment buffer solution).

26. The method of claim 25, wherein the buffer solution comprises phosphate.

27. (canceled)

28. The method of claim 25, wherein the collagen and silk polypeptide composite structure is exposed to the buffer solution for a time period of between 1 min and 24 hours.

29.-41. (canceled)

42. A collagen and silk polypeptide composite structure obtainable by the method of claim 1.

43. The collagen and silk polypeptide composite structure of claim 42, wherein the structure is a 3D collagen and silk polypeptide composite structure, a collagen and silk polypeptide laminate, a collagen and silk polypeptide composite scaffold.

44. A collagen and silk polypeptide composite structure comprising
collagen and
a silk polypeptide dispersed therein,
wherein the collagen and the silk polypeptide are directly linked/associated with each other (without (the need of) a separate cross-linker).

45. The collagen and silk polypeptide composite structure of claim 44, wherein the collagen and the silk polypeptide are present at a ratio of between 40:60 and 60:40 in the structure.

46. The collagen and silk polypeptide composite structure of claim 44, wherein the collagen is comprised in form of collagen fibrils and/or the silk polypeptide is comprised in form of silk polypeptides particles in the structure.

47.-49. (canceled)

50. Implant or wound closure material comprising or consisting of the collagen and silk polypeptide composite structure of claim 44.

51.-59. (canceled)

60. A system for carrying out the method of claim 1, wherein the system comprises a processor capable of executing machine readable instructions when the machine-readable instructions are loaded into the system.

61. A computer program comprising machine-readable instructions which when loaded into the system of claim 60 cause the system to carry out the method of claim 1.
